SEQUENCE LISTING (26 sequences)

SEQ ID NO: 1 - DNA, 22 bp, Artificial sequence
  Synthetic primer
accctcatcaatggcctgtgga

SEQ ID NO: 2 - DNA, 20 bp, Artificial sequence
  Synthetic primer
catgggctttgacccttggg

SEQ ID NO: 3 - DNA, 19 bp, Artificial sequence
  Synthetic primer
atcatgggaaacccccgga

SEQ ID NO: 4 - DNA, 25 bp, Artificial sequence
  Synthetic primer
gggtgaattccttggcaaaatgagt

SEQ ID NO: 5 - DNA, 24 bp, Artificial sequence
  Synthetic primer
tctgctatcatgggtaatgccaaa

SEQ ID NO: 6 - DNA, 19 bp, Artificial sequence
  Synthetic primer
gaaggcagcctgtgcagcg

SEQ ID NO: 7 - DNA, 20 bp, Artificial sequence
  Synthetic primer
gctgcctgggggaagattgg

SEQ ID NO: 8 - DNA, 20 bp, Artificial sequence
  Synthetic primer
gggtgaaatcggcagggtgg

SEQ ID NO: 9 - DNA, 24 bp, Artificial sequence
  Synthetic primer
ggatattttgaccattcgatcatg

SEQ ID NO: 10 - DNA, 25 bp, Artificial sequence
  Synthetic primer
ccagtctgctgctctgtaacaatct

SEQ ID NO: 11 - DNA, 21 bp, Artificial sequence
  Synthetic primer
catcaacaagcccaggttcaa

SEQ ID NO: 12 - DNA, 20 bp, Artificial sequence
  Synthetic primer
ctcacccatctgttggtcga

SEQ ID NO: 13 - DNA, 19 bp, Artificial sequence
  Synthetic primer
ccggaatctccagggatga

SEQ ID NO: 14 - DNA, 23 bp, Artificial sequence
  Synthetic primer
tcttttgtcctgtctgctggtct

SEQ ID NO: 15 - DNA, 21 bp, Artificial sequence
  Synthetic primer
catcaacaagcccaggttcaa

SEQ ID NO: 16 - DNA, 20 bp, Artificial sequence
  Synthetic primer
aggtgaggctaccgcagagt

SEQ ID NO: 17 - DNA, 19 bp, Artificial sequence
  Synthetic primer
ctgcatcaacaagcccagg

SEQ ID NO: 18 - DNA, 19 bp, Artificial sequence
  Synthetic primer
gggcccctagaccaggaaa

SEQ ID NO: 19 - DNA, 24 bp, Artificial sequence
  Synthetic oligonucleotide
ccctgaggacacaggtcagccttg

SEQ ID NO: 20 - DNA, 22 bp, Artificial sequence
  Synthetic oligonucleotide
cagccttgaccaatgactttta

SEQ ID NO: 21 - DNA, 28 bp, Artificial sequence
  Synthetic oligonucleotide
gccttgccttgaccaatagccttgacaa

SEQ ID NO: 22 - DNA, 22 bp, Artificial sequence
  Synthetic oligonucleotide
tagggttggccaatctactccc

SEQ ID NO: 23 - DNA, 25 bp, Artificial sequence
  Synthetic oligonucleotide
ccatgaggaccacgggtcaggctga

SEQ ID NO: 24 - DNA, 23 bp, Artificial sequence
  Synthetic oligonucleotide
tcaggctgaccaatggcttcaaa

SEQ ID NO: 25 - DNA, 34 bp, Artificial sequence
  Synthetic oligonucleotide
cccagactctcttgaccaatagcctcagagtcct

SEQ ID NO: 26 - DNA, 23 bp, Artificial sequence
  Synthetic oligonucleotide
tggtaagggccaatctgctcaca

(19) United States
(12) Patent Application Publication
(10) Pub. No.: US 2008/0008651 A1
(43) Pub. Date: Jan. 10, 2008

(54) Screening Methods and Transgenic Animals for the Treatment of Beta-Globin Related Disease and Conditions

(75) Inventor: James D. Engel, Ann Arbor, MI (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE REGENTS OF THE UNIVERSITY OF MICHIGAN, Ann Arbor, MI (US)

(21) Appl. No.: 11/754,197

(22) Filed: May 25, 2007

Related U.S. Application Data
(60) Provisional application No. 60/808,364, filed on May 25, 2006.

(51) Int. Cl. A61K 49/00; A61P 7/00; G01N 33/53

(52) U.S. Cl. 424/9.1; 435/7.1; 514/789

(57) ABSTRACT

The orphan nuclear receptors TR2 and TR4 together constitute the DNA binding core of the 540 kDa DRED complex, a putative repressor of the human embryonic ε- and fetal γ-globin genes. Here the functional consequences of TR2 and TR4 germ line loss of function were examined, transgenic gain of function and dominant negative gain of function on human and murine β-type globin gene expression throughout development. ε-globin transcription responded in a manner consistent with the hypothesis that TR2/TR4 is a constitutive erythroid ε-globin repressor. In contrast, parallel experiments show that TR2/TR4 is a definitive stage-selective γ-globin repressor. This developmental stage-specific, gene-selective repression of the ε- and γ-globin genes by TR2/TR4 establishes, when considered in concert with the competition hypothesis, a coherent molecular rationale for hemoglobin switching (temporally specific, sequential activation of all the β-type globin genes) during vertebrate development.

### BACKGROUND OF THE INVENTION

The human β-globin locus is larger than 70 kbp, and is composed of five globin genes that are spatially arranged and developmentally expressed in the order (from 5′ to 3′): ε-(embryonic), Gγ- and Aγ- (fetal) and δ- and β-globin (adult). The embryonic ε-globin gene is expressed during the first 6 to 8 weeks of human gestation in erythroid cells produced in the yolk sac, the major site of blood production in the early embryo (primitive erythropoiesis). The first switch in β-globin subtypes results in silencing of the ε-globin gene and concomitant activation of the fetal γ-globin genes when definitive erythropoiesis ensues and the site of erythropoiesis shifts to the fetal liver. Gradually, beginning at around the time of birth, the second switch from γ- to β-globin transcription occurs as the site of hematopoiesis shifts once more from the fetal liver to the adult bone marrow and spleen (Stamatoyannopoulos and Grosveld, 2001).

From genetic analyses of transgenic mice harboring mutated human β-type globin loci, two nonexclusive mechanisms for β-globin switching during development have been postulated: one is regulation by local regulatory sequences located in the promoter regions of each globin gene (autonomous gene control) (Magram et al., 1985) (Townes et al., 1985) (Dillon and Grosveld, 1991; Raich et al., 1990), and the other is competition among the globin genes (Choi and Engel, 1988) for activation by the locus control region (LCR), an element required for the high-level expression of each globin gene (Behringer et al., 1990; Enver et al., 1990). In a competitive model, the gene closer to LCR should have a higher probability for interaction with the LCR and hence be more abundantly expressed, unless the gene is autonomously silenced (Hanscombe et al., 1991) (Tanimoto et al., 1999). An autonomous control mechanism has been shown to play a major role in silencing of the human embryonic ε- and fetal γ-globin genes in definitive erythroid cells while competitive gene control has been shown to play a major role in the silencing of the human adult β-globin gene at the embryonic and fetal stages (Tanimoto, 1999). However, the molecular basis of these regulatory mechanisms is still only incompletely defined.

In analyzing possible autonomous silencing mechanisms governing transcriptional regulation of the ε- and γ-globin genes, direct repeat (DR) elements (AGGTCA repeats), consensus binding sites for non-steroidal nuclear receptors, were identified in the proximal promoters of both the ε- and γ-globin genes (FIG. 1A). Mutation of the DR sequences in the ε-globin promoter led to its de-repression in definitive erythroid cells of transgenic mice (Filipe et al., 1999; Tanimoto et al., 2000). In the human hematologic condition known as hereditary persistence of fetal hemoglobin (HPFH), the fetal γ-globin gene continues to be abundantly transcribed during adulthood, leading to elevated synthesis (up to 30%) of γ-globin in adult erythrocytes which normally have only very low levels (usually <1%) of tetrameric hemoglobin F (α₂γ₂) (Stamatoyannopoulos and Grosveld, 2001). Analyses of HPFH mutations have provided key insights into the regulatory mechanisms controlling γ-globin transcription. HPFH mutations include both small and large deletions in the β-globin locus, as well as point mutations in both γ-globin promoters. Of sixteen different mutations identified in the Gγ or Aγ promoters that are associated with HPFH, six are located within DR elements (Huisman et al., 1997). Introduction of artificial or naturally occurring mutations into the DR element leads to derepression of γ-globin transcription in transgenic mice (Berry et al., 1992) (Omori et al., 2005). These observations suggested an essential role for the DR elements in both ε- and γ-globin gene silencing in definitive erythroid cells.

TR2 and TR4 are two closely related members of the nuclear receptor superfamily and have diverse biological functions. They form homodimers or heterodimers and bind to AGGTCA direct repeats (DR) separated by a 0 to 6 nucleotide spacer (DR0-DR6), and either activate or repress their target genes depending on the context of their recognition sequences or on cellular conditions (Lee et al., 2002). TR2 and TR4 share common functions in a variety of biological processes elicited by key signaling molecules such as thyroid hormone, ciliary neurotrophic factor, and retinoic acid. Recent gene ablation studies have revealed some in vivo functions of these receptors. Mice lacking TR2 are viable and show no overt defects (Shyr et al., 2002), but TR4 germline mutants display reproductive and neurological phenotypes (Mu et al., 2004; Collins et al., 2004; Chen et al., 2005).

A novel sequence-specific DNA binding factor, DRED (direct repeat erythroid definitive), and an orphan nuclear receptor, COUP-TFII, have been shown to bind to the DR elements in the ε- and γ-globin promoters in vitro, and both have been implicated in repression of these genes in definitive erythroid cells (Filipe et al., 1999) (Tanimoto et al., 2000). In order to characterize DRED, DRED was purified from nuclear extracts of a murine definitive erythroid cell line, MEL, and found that DRED is a 540 kDa complex that contains a heterodimer of the TR2 and TR4 orphan nuclear receptors as its DNA binding scaffold (Tanabe et al., 2002). These data indicated that TR2 and TR4 are the DNA binding core of the larger DRED complex, and hence that DRED could play a key role in repressing ε- and γ-globin transcription in definitive erythroid cells. Thus, there remains a need in the art to identify modulators of TR2, TR4 and the TR2/TR4 heterodimer (and the DRED complex) and to determine the effects of the identified modulators on erythroid differentiation.

### SUMMARY OF THE INVENTION

The genetic analyses of TR2 and TR4, and the loss and gain of TR2 and TR4 function effects on the regulation of β-type globin transcription using the Tr2 and Tr4 knockout mice as well as transgenic mice in which wild-type or dominant negative mutant receptors were forcibly expressed in erythroid cells is disclosed herein. As a consequence of erythroid-specific TR2/TR4 forced expression, both the mouse embryonic εy- and human embryonic ε-globin genes were repressed in both primitive and definitive erythroid cells. Surprisingly however, TR2/TR4 transgenic expression simultaneously resulted in activation of the mouse embryonic βh1- as well as its ortholog, the transgenic human fetal γ-globin gene. Following enforced transgenic expression of a dominant negative TR4 mutant, the human ε-globin gene was activated in both primitive and definitive erythroid cells, consistent with the properties conferred by the wild-type transgenic receptors. In contrast, the human γ-globin gene was activated only in definitive erythroid cells by the TR4 dominant negative mutant. These data indicate that TR2 and TR4 together constitute a stage-independent repressor of the human ε-globin gene, and that they also function as a definitive stage-specific repressor of the human fetal γ-globin genes. As such, the data presented herein constitute the first direct evidence demonstrating that specific transacting factors control gene autonomous silencing of the embryonic and fetal β-type globin genes through well defined cis-regulatory elements in their promoters, and indirectly suggests that TR2 and TR4 may be unusually attractive targets for therapeutic intervention in the treatment of sickle cell disease. This temporally specific, gene-selective repression of the human embryonic and fetal β-type globin genes by TR2 and TR4 provides a basis for explaining the gene autonomous and sequential silencing of the ε- and γ-globin genes during embryonic development, and in concert with the competition hypothesis provides a molecular explanation for how globin gene switching during human development ensues.

Methods are provided for identifying a compound that stimulates expression of a gene product in a definitive erythroid cell, the method comprising the step of measuring expression of a gene product in the absence and presence of a candidate substance, TR2, and TR4 with a γ-globin gene promoter sequence, wherein said gene product is encoded by a polynucleotide operatively linked to the γ-globin gene promoter sequence and the TR2 and TR4 form a heterodimer and bind the promoter sequence in the absence of the candidate substance. An increase in gene product expression in the presence of the candidate substance, compared to gene product expression in the absence of the candidate substance, identifies the candidate substance as a compound that stimulates expression of the gene product. In one aspect, the candidate substance is selected from the group consisting of a small molecule, a peptide, a polypeptide, a synthetic compound, and a naturally-occurring compound. In other aspects, the candidate substance is an antibody or an antigen binding fragment or derivative thereof.

Methods are also provided for identifying a compound that stimulates expression of a gene product in a definitive erythroid cell comprising the step of measuring expression of a gene product in the absence and presence of a candidate substance and a TR2/TR4 heterodimer with a γ-globin gene promoter sequence, wherein the gene product is encoded by a polynucleotide operatively linked to the γ-globin gene promoter sequence and the TR2/TR4 heterodimer binds the promoter sequence in the absence of the candidate substance. An increase in gene product expression in the presence of the candidate substance, compared to gene product expression in the absence of the candidate substance, identifies the candidate substance as a compound that stimulates expression of the gene product. In one aspect, the candidate substance is selected from the group consisting of a small molecule, a peptide, a polypeptide, a synthetic compound, and a naturally-occurring compound. In other aspects, the candidate substance is an antibody or an antigen binding fragment or derivative thereof.

In one embodiment, the TR2/TR4 heterodimer is part of a direct repeat erythroid definitive (DRED) protein complex (DRED/TR2/TR4) and the increase in gene product expression is associated with a decrease in DRED/TR2/TR4 binding to all or part of a γ-globin gene promoter to a degree sufficient to inhibit gene transcription.

In another embodiment, the TR2/TR4 heterodimer is independent of a direct repeat erythroid definitive (DRED) protein complex and the increase in gene product expression is associated with an increase in TR2/TR4 heterodimer binding to all or part of a γ-globin gene promoter to a degree sufficient to increase gene transcription.

Also provided are methods of identifying a compound that inhibits formation of a TR2/TR4 heterodimer, the methods comprising the step of measuring TR2/TR4 heterodimer formation in the absence and presence of a candidate substance. A decrease in the formation of a TR2/TR4 heterodimer in the presence of the candidate substance, compared to heterodimer formation in the absence of the candidate substance, identifies the candidate substance as a compound that inhibits the formation of the TR2/TR4 heterodimer. In one aspect, the candidate substance is selected from the group consisting of a small molecule, a peptide, a polypeptide, a synthetic compound, and a naturally-occurring compound. In other aspects, the candidate substance is an antibody or an antigen binding fragment or derivative thereof.

In some embodiments, the methods described herein are carried out ex vivo. In other embodiments, the methods described herein are carried out in vivo.

In some embodiments, the gene product is γ-globin. In other embodiments, the gene product is a polypeptide encoded by a polynucleotide, which does not encode γ-globin, operatively-linked to the γ-globin gene promoter.

In yet another embodiment, a compound that stimulates expression of the gene product identified by a method described herein or a compound that inhibits formation of s TR2/TR4 heterodimer, as well as compositions and pharmaceutical compositions comprising the compound, are contemplated.

In still another embodiment, methods of treating a disorder associated with aberrant globin expression comprising the step of administering a therapeutically effective amount of a compound that stimulates the expression of a gene product are contemplated. “Aberrant globin expression” is expression of a globin gene product that gives rise to (or is associated with) a hematological disorder.

In another embodiment, methods of treating a disorder associated with aberrant globin expression comprising the step of contacting a TR2/TR4 heterdimer with an inhibitor that prevents binsing of the TR2/TR4 heterodimer to a γ-globin gene promoter sequence are contemplated. In yet another embodiment, methods of treating a disorder associated with aberrant globin expression comprising the step of contacting TR2 and/or TR4 with an inhibitor that prevents heterodimer formation are contemplated. In one aspect, the disorder is associated with expression of an abnormal 13 globin gene product. In one aspect the 13 globin gene product is 13 globin. In another aspect, the disorder is sickle cell anemia. In yet another aspect, the disorder is 13-thalessemia.

In still another embodiment, methods of treating sickle cell anemia comprising the step of administering to an individual in need a therapeutically effective amount of a compound that selectively stimulates expression of γ-globin and reduces expression of β-globin are contemplated.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. Additional features and variations of the invention will be apparent to those skilled in the art from the entirety of this application, and all such features are intended as aspects of the invention.

### BRIEF DESCRIPTION OF THE FIGURES

FIG. 1. DRED binding to the DR Elements of Human and Mouse Embryonic and Fetal β-type Globin Genes. (A) Alignment of the promoter sequences of human and mouse β-type globin gene orthologues. Nucleotides in potential DR elements (indicated by horizontal arrows) are shown in bold letters, where those matching the consensus sequence for nuclear receptor binding are indicated with upper case letters. The numbers adjacent to each potential DR element represents the nM Ki determined for that binding site. (B) EMSA competitive binding assays using 1.1 nM 32P-labeled ε distal DR probe, and 20 or 200 nM (18- or 180-fold molar excess) unlabeled competitor oligonucleotides. The arrowhead indicates the position of the authentic DRED complex. The relative abundance of bound probe is shown at the bottom of each lane (the bound probe with no added competitor set at 100%). -, no competitor. (C) 10 μg of pEF-BOS expression vector driving TR2 or TR4 cDNAs (Tanabe et al., 2002) was transfected separately or together into 293T cells for nuclear extract preparation and EMSA (top panel) and Western blotting with an anti-TR2 (middle) or anti-TR4 (bottom) antiserum. 10 μg of a CMV expression vector driving transcription of putative dominant negative (dn) TR2 or TR4 mutants was also transfected into 293T cells. The arrowhead indicates the mobility of authentic DRED complex from MEL cell nuclear extract.

FIG. 2. Mouse β-type Globin Gene Expression in Tr2 or Tr4 Null Mutant Mice. Expression of embryonic εy-, βh1- and adult βmajor-globin genes in the 10.5 dpc yolk sac and 13.5 dpc fetal liver of Tr2 or Tr4 null mutant mice was determined by semi-quantitative RT-PCR. For yolk sac analysis, radioactive signals for εy (13 cycles), βh1 (13 cycles), or βmajor (14 cycles) PCR products were quantified and normalized to α-globin mRNA levels co-amplified in the same sample tubes. For fetal liver analysis, εy (13 cycles) and βmajor (11 cycles) amplicons were normalized to co-amplified α-globin abundance, and βh1 product (19 cycles) was separately normalized to α-globin amplification (13 cycles). The relative expression levels of each globin gene normalized to wild-type (+/+) littermates (100%) are graphically depicted with standard deviations (S.D.). The number of animals of each genotype analyzed was 2 to 7 for the yolk sac analysis, and 5 to 8 for the fetal liver analysis. *P=0.08, **P=0.03 by Student t-test.

FIG. 3. Relative mRNA Abundance of the TR2 or TR4 Transgenes. The abundance of endogenous (open bars) or transgenic (shaded) TR2 (upper panel) and TR4 (lower) mRNAs in the 14.5-dpc fetal liver of the transgenic mice overexpressing TR2, TR4, or both, or expressing the dominant negative (dn) TR4 mutant, was determined by reverse transcription of mRNA followed by real-time PCR, and normalized to the abundance of endogenous GATA-1 mRNA (set at 100%). Data represent the averages ±S.D. of 2 to 3 fetal liver samples from each transgenic line or 14 wild-type fetal livers.

FIG. 4. Mouse β-type Globin Gene Expression in Transgenic Mice Forcibly Expressing TR2 or TR4. (A) Expression of the embryonic εy-, βh1- and adult βmajor-globin genes in the 9.5 dpc yolk sac of TR2, TR4, or TR2/TR4 transgenic mice was determined by semi-quantitative RT-PCR. The abundance of εy (13 cycles), βh1 (13 cycles) or βmajor (15 cycles) PCR products were quantified and normalized to co-amplified α-globin quantity. The averages ±S.D. for the relative expression level of each globin gene normalized by the wild-type (Wt) littermates (set at 100%) are graphically depicted. The number of animals of each genotype analyzed was 3 to 6. (B) Expression of γy, βh1 and βmajor genes in the 14.5 dpc fetal liver. εy (16 cycles) or βh1 (19 cycles) were normalized to α-globin abundance, amplified separately (11 cycles), or βmajor amplicons (11 cycles) were normalized to co-amplified α-globin abundance. The averages ±S.D. for the relative expression levels normalized to Wt littermates are shown as in A. The number of animals of each genotype analyzed was 3 to 6. (C) Genetic synergy between TR2 and TR4. Expression of the εy and βh1 genes in the 14.5-dpc fetal liver of transgenic fetuses generated by intercrosses between TR2 and TR4 transgenic lines were determined as in B. The number of animals of each genotype analyzed was 3 to 5. (D) Expression of βh1 and βmajor mRNAs in the adult spleen. βh1 (27 cycles) amplification was normalized to α-globin mRNA abundance, amplified separately (13 cycles), while βmajor abundance (13 cycles) was determined relative to co-amplified α-globin abundance. The averages ±S.D. for the relative expression levels normalized to Wt littermates are shown as in A. The number of animals of each genotype analyzed was 2 to 3. *P=0.07, **P<0.05, ***P<0.005 by t-test.

FIG. 5. Time-course of Mouse β-type Globin mRNA Accumulation and Midembryonic Anemia in TR2/TR4 Transgenic Mice. (A) Expression of embryonic εy, βh1 and adult βmajor globin genes in the yolk sac and fetal livers of TgTR2/TR4 mice (line 1) and wild-type littermates from 9.5 to 15.5 dpc was determined by semi-quantitative RT-PCR. For yolk sac analysis, amplification of εy (13 cycles), βh1 (13 cycles) or βmajor (11 to 15 cycles) were quantified and normalized to co-amplified α-globin signals. For fetal liver analysis, εy (16 cycles) or βh1 (16 cycles) amplicons were normalized to α-globin, amplified separately (13 cycles), while βmajor (11 cycles) was normalized to co-amplified α-globin. The averages ±S.D. for the relative expression levels of each globin gene are graphically depicted. Note that the scales for εy and βh1 abundances differ between the yolk sac and fetal liver data. The number of animals analyzed of each genotype was 2 to 3. *P<0.05, **P<0.005 by t-test. (B) Yolk sacs and embryos of 11.5 dpc TgTR4 (line 2) embryos and a wild-type (Wt) littermate. (C) Quantification of red blood cells recovered from 10.5 to 12.5 dpc TgTR2/TR4 (line 2) embryos and their wild-type littermates. Data are the averages ±S.D. of 5 to 8 embryos per time point.

FIG. 6. Altered Human β-type Globin Gene Transcription in TR2/TR4 Transgenic Mice. (A) Expression of human embryonic ε-, fetal γ-, and adult β-globin genes in the 10.5 dpc yolk sac, 15.5 dpc fetal liver or adult spleen of transgenic mice bearing a wild type human β-globin YAC transgene (Tanimoto et al., 1999) with (+) or without (−) TgTR2/TR4 was determined by semiquantitative RT-PCR, as described in Material and Methods. The averages ±S.D. for relative expression levels of each human globin gene normalized to that of littermates without TgTR2/TR4 (−, set at 100%). Two to five animals from 1 or 2 litters of each genotype were examined. (B) Expression of the human embryonic ε-globin gene in the 10.5 dpc yolk sac of transgenic mice bearing the DR mutant human β-globin YAC transgene, Bepsi (Tanimoto et al., 2000), in the presence (+) or absence (−) of TgTR2/TR4 (line 2) was determined as described in A, and normalized to the expression in mice bearing the wild-type β-globin YAC without TgTR2/TR4 (100% in A). Three fetuses of each genotype were examined. (C) γ-globin cDNAs from the 15.5 dpc fetal liver of transgenic mice bearing a wild-type or mutDR (Omori et al., 2005) human β-globin YAC transgene either in the presence (+) or absence (−) of TgTR2/TR4 (line 2) were amplified by PCR as in A, and then digested with PstI to determine the Gγ/Aγ molar ratio (Omori et al., 2005). The averages ±S.D. for the relative expression levels of Gγ- and Aγ-globin were normalized to total γ-globin mRNA accumulation in mice bearing the wild-type β-globin YAC transgene (-TgTR2/TR4, set at 100% in A) are graphically depicted. Three fetuses of each genotype were examined. (D) Expression of the human ε-, γ-, and β-globin genes in the yolk sac and fetal liver of transgenic mice bearing the wild type human β-globin YAC, with or without TgTR2/TR4 (line 1), from 9.5 to 16.5 dpc was determined by semi-quantitative RT-PCR, as above. The averages ±S.D. for the relative mRNA abundance of each globin gene are graphically depicted. Note that the scales for the ε and γ mRNA accumulation differ between the yolk sac and fetal liver. Two to five animals from 1 or 2 litters of each genotype were examined. *P<0.05, **P<0.005 by t-test.

FIG. 7. A Dominant Negative TR4 Mutant Transgene Induces Embryonic and Fetal Globin mRNAs. (A) Alignment of the amino acid sequences of the DNA binding domains of TR2, TR4, and RXRα. The residues mutated in the putative dominant negative TR2 or TR4 mutants are shown at the top. Residues in RXRα that make base or phosphate contacts are indicated by triangles below the panel (Zhao et al., 2000). (B) 4 μg of a CMV expression vector bearing wild type TR2 (left panel) or TR4 (right) cDNAs was transfected into 293T cells with or without co-transfection of 4 or 16 μg of a CMV expression vector bearing the putative dominant negative TR2 or TR4 mutants, followed by nuclear extract preparation and EMSA. The relative abundance of DR probe bound to the various nuclear extracts is indicated at the bottom of each lane (bound probe in the absence of the dominant negative mutants was set at 100%). (C) Expression of human ε- and γ-globin genes in the 8.5 to 12.5 dpc yolk sac of transgenic mice bearing the wild type human β-globin YAC, with or without intercrossed TgdnTR4, was determined by semi-quantitative RT-PCR. Products for ε-(12-13 cycles) or γ-globin transcripts (12-13 cycles) were quantified and then normalized to the abundance of co-amplified mouse α-globin. The averages ±S.D. for the relative expression of each globin gene are graphically depicted. One to five embryos from 1 to 2 litters of each genotype were examined. (D) Expression of the human ε- and γ-globin genes in 14.5 dpc fetal livers of transgenic mice bearing the wild type human β-globin YAC, with (+) or without (−) the intercrossed TgdnTR4, was determined as in FIG. 6A. Data are the averages ±S.D. for the relative expression of each globin gene in 7 fetuses with, and 3 without, TgdnTR4. *P=0.058, **P<0.05 by t-test.

FIG. 8. A Hypothetical Model for the Role of TR2/TR4 in Developmental Stage-specific Silencing of the Human ε- and γ-Globin Genes. In primitive erythroid cells (top), TR2/TR4 collaboratively forms DRED, a complex of TR2/TR4 and other (limiting) co-repressors, and DRED (because of higher affinity for the ε-globin promoter DR sites, or because of its activity/abundance at that stage) represses ε-globin transcription. TR2/TR4 exerts little or no net effect on the γ-globin gene, since the activities of the DRED complex and a counteracting activator complex containing TR2/TR4 are balanced on the γ-globin gene DR element in primitive erythroid cells. In definitive erythroid cells (bottom), the activity/abundance of DRED increases, allowing it to now repress γ-globin synthesis from the (lower affinity) DR sites in the γ-globin promoters.

### DETAILED DESCRIPTION OF THE INVENTION

The data described herein demonstrate a temporally specific, gene-selective positive and negative regulation of mammalian embryonic and fetal β-type globin genes by TR2 and TR4. TR2 and TR4 can function as transcriptional activators as well as repressors in the regulation of other target genes, depending on the context of recognition sequences and cellular conditions (Lee et al., 2002) or their interactions with various co-regulators [with co-repressor TRA16 (Yang et al., 2003), histone deacetylases 3 and 4 (Fantoni et al., 1967), or RIP140, which can function as a co-activator (Cavailles et al., 1995) or co-repressor (Lee et al., 1998)]. It is therefore contemplated that these dual regulatory activities of TR2/TR4 in the human β-globin locus are achieved by sequence context- and developmental stage-dependent interaction with differential co-regulators (Glass and Rosenfeld, 2000). Sequence differences within the DR elements or in the sequences surrounding them may be responsible for contextual effects of TR2 and TR4 regulation on the embryonic and fetal β-type globin genes. The underlying molecular mechanisms for such differential effects may include allosteric regulation of TR2/TR4 binding specificity for co-regulators by the bound DNA sequences (Scully et al., 2000), the differential affinity of the DR elements for TR2/TR4 complexed with different co-regulators, or the differential availability of co-regulating transcription factors that bind to sequences in the vicinity of the DR elements. Any or all of these mechanisms may lead to differential recruitment of activating or repressing co-regulators to different promoters. Although ligand molecules for TR2 or TR4 have not been identified, it is also possible that the temporally specific or geneselective activity of TR2/TR4 on the β-type globin genes reported here may be controlled in part by the differential availability of a small molecule ligand. This hypothesis implies that β-type globin gene switching may be, in part, controlled by an inductive extracellular signaling molecule, which would provide a non-cell autonomous basis for developmental stage specificity and synchronicity of globin gene switching.

The experiments conducted herein examining the consequences of the dnTR4 mutant mice (see Example 6), as well as the Tr2 or Tr4 null mutant mice (See Example 2), indicate a negative regulatory role for TR2 and TR4 on murine βH1 and human γ-globin transcription in the fetal liver. However, forced expression of TR2/TR4 in definitive erythroid cells demonstrated only a seemingly contradictory (activator) function on the human γ-globin genes. It is contemplated that these paradoxical results are due to the limited abundance or activity of co-repressors that can interact with the artificially (10-fold or more) abundant TR2/TR4 molecules in the erythroid cells of these transgenic mice, thereby possibly leading to preferential formation of either sterile repressor, or alternatively, TR2/TR4-mediated transcriptional activator, binding to the βh1 or γ-globin gene DR sites.

Control of β-Type Globin Gene Switching Mediated by TR2/TR4

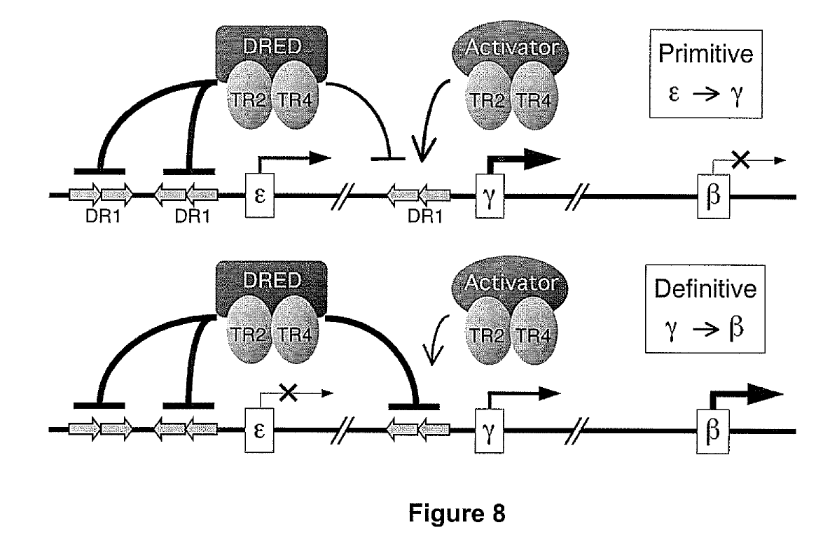

In the primitive erythroid cells of the yolk sac, TR2/TR4 binds to the DR elements of the ε-globin gene as a component of the DRED complex, thereby repressing ε-globin transcription (FIG. 8). At the same time, TR2/TR4 exerts little or no effect on the γ-globin gene DR sites at this developmental stage; these data are also consistent with the results of the dnTR4 mutant analysis. These observations are consistent with the lack of any discernable effect on γ-globin DR site-specific mutant transcription (in Tg^{mutDR}) in primitive erythroid cells (Omori et al., 2005). Based on the observed activator as well as repressor activity of TR2/TR4 on the γ gene, it is contemplated that both the DRED complex and a counteracting complex that consists of TR2/TR4 and unknown co-activators, can bind to the γ-globin gene DR element, and that the activities of these two (activator and repressor) complexes are balanced during the primitive stage, thus exerting little or no net effect on transcription. In one aspect, these differential activities of TR2/TR4 on the ε- and γ-globin gene promoters constitute a molecular basis for selective, gene-autonomous repression that enables the ε- to γ-globin switch at the fetal liver stage of erythropoiesis.

In one aspect, in definitive erythroid cells of the fetal liver, the activity of DRED on the γ-globin promoter surpasses the activity of the (primitive erythroid) activator complex, either by diminishing the activity of components of the activator complex or by elevated DRED activity, and thereby promotes repression of both the ε- and γ-globin genes in an autonomous manner, thus enabling the subsequent γ- to β-globin switch. Although ligands for TR2 and TR4 have not been identified, the activity of TR2/TR4 can mediated by differential availability of small molecule ligands, which would provide a non-cell-autonomous basis for the developmental stage specificity and synchronicity of globin gene switching.

Genetic analyses examining transgenic mice harboring mutated human β-type globin loci have revealed that silencing of the embryonic and fetal β-type globin genes is initiated by gene-autonomous mechanisms (Magram et al., 1985) (Townes et al., 1985) (Raich et al., 1990) (Dillon and Grosveld, 1991), although the relative contribution of autonomous control versus competitive silencing (Tanimoto et al., 2000) is unresolved. In analysis of possible cis-regulatory elements governing transcription of the ε- and γ-globin genes, multiple silencing elements have been proposed to lie in the proximal or distal regions of those promoters (Raich et al., 1992) (Raich et al., 1995) (Peters et al., 1993) (Li, J. et al., 1998; Li, Q. et al., 1998; Tanimoto et al., 2000), including those identified through the analysis of the HPFH mutations (Berry et al., 1992; Li et al., 2001) (Ronchi et al., 1996) (Omori et al., 2005). However, the identity of the trans-acting regulatory factors that recognize such potential cis-regulatory elements has been elusive.

Taken together, the data presented herein represents the first genetic evidence demonstrating that a trans-acting factor directly controls gene-autonomous silencing of the embryonic and fetal β-type globin genes through a well defined cis-regulatory element in their promoters.

TR2/TR4 as a Target for Therapeutic Intervention in Sickle Cell Disease

Sickle cell disease is caused by a missense mutation in the adult β-globin gene and affects millions of people worldwide (Bunn, 2001) (Stuart and Nagel, 2004). Tetrameric hemoglobin incorporating the mutant β-globin polypeptide polymerizes (when deoxygenated) and causes erythrocytes to acquire a stiff sickle shape. Sickle erythrocytes are subject to premature destruction, and prone to occlude blood flow, causing vascular damage in multiple organ systems. Based on biochemical evidence demonstrating the inhibitory effects of γ-globin on polymerization of deoxygenized sickle hemoglobin in vitro, therapeutic agents that increase γ-globin production are widely expected to benefit sickle cells patients (Steinberg and Rodgers, 2001). In fact, previous clinical studies have shown that sickle cell patients that expressed higher levels of fetal hemoglobin experience milder disease and decreased mortality (Platt et al., 1994).

The data herein showing that the γ-globin gene is activated in the adult spleen by TR2/TR4 forced expression provides direct evidence that TR2/TR4 serves as a target for therapeutic intervention in treating sickle cell disease: pharmacological stimulation of the activator function, or alternatively inhibition of the repressor function of TR2/TR4 in definitive erythroid cells would induce γ-globin synthesis and thereby ameliorate the disease. To develop therapeutics that modulate TR2/TR4 activity, identification of natural ligand(s) of TR2 and TR4 is contemplated to facilitate the rational design of drugs that enhance the activator function or block the repressor function of TR2/TR4. Identification of co-regulators of TR2/TR4 in the larger DRED complex (Tanabe et al., 2002) is contemplated for developing new therapeutics, because the interfaces between TR2/TR4 and co-regulators constitute additional targets for drug design that will selectively enhance the interactions between TR2/TR4 and coactivators, or block effective interactions with co-repressors.

Assaying for Modulators of Erythroid Differentiation

The present invention has several aspects, one of which is identifying modulators of erythroid differentiation. In one aspect, the invention provides methods of screening for modulators of TR2, TR4 and TR2/TR4 heterodimer protein activity, the effects of TR2, TR4 and TR2/TR4 heterodimer in the presence and absence of the candidate substance and comparing such results. Such screening techniques are useful in the general identification of a compound that will modulate erythroid differentiation in a cell, with such compounds being useful as therapeutic agents. For example, those compounds that decrease the activity or expression of TR2, TR4 and TR2/TR4 heterodimer are useful in the treatment of various hemoglobinopathies because it is shown herein that the TR2/TR4 heterodimer functions as a repressor of the fetal γ-globin gene, and thus plays a significant role in erythroid differentiation. Compounds that prevent TR2/TR4 heterodimer formation are also contemplated.

In the screening embodiments, methods are provided for identifying a compound that stimulates expression of a gene product in a definitive erythroid cell, the method comprising the step of measuring expression of a gene product in the absence and presence of a candidate substance, TR2, and TR4 with a γ-globin gene promoter sequence, wherein said gene product is encoded by a polynucleotide operatively linked to the γ-globin gene promoter sequence and the TR2 and TR4 form a heterodimer and bind the promoter sequence in the absence of the candidate substance. An increase in gene product expression in the presence of the candidate substance, compared to gene product expression in the absence of the candidate substance, identifies the candidate substance as a compound that stimulates expression of the gene product.

In one embodiment, methods are provided for identifying a compound that alters TR2, TR4 or TR2/TR4 heterodimer protein expression or activity in cells that either naturally express TR2, TR4 or TR2/TR4 heterodimer protein or have been engineered to express the TR2, TR4 or TR2/TR4 heterodimer protein. Methods of transfecting cells with a polynucleotide that encodes a given protein are routine in the art.

An alteration in the TR2, TR4 or TR2/TR4 heterodimer protein activity and/or expression in the presence of the candidate substance will indicate that the candidate substance is a modulator of the activity and/or expression of TR2, TR4 or TR2/TR4 heterodimer.

To identify a candidate substance as being capable of modulating TR2, TR4 or TR2/TR4 heterodimer protein activity, one measures TR2, TR4 or TR2/TR4 heterodimer protein activity in the absence of the added candidate substance. A candidate inhibitory/stimulator substance is then added to the cell and the TR2, TR4 or TR2/TR4 heterodimer protein activity, expression or other characteristic output is determined in the presence of the candidate inhibitory/stimulatory substance. A candidate substance which is inhibitory would decrease the TR2, TR4 or TR2/TR4 heterodimer protein activity or expression (and in turn stimulate production of a gene product in an erythroid cell), relative to the same parameter in its absence, whereas a stimulator will increase such an activity or expression (and in turn inhibit production of a gene product in an erythroid cell), relative to the parameter in its absence.

A. Candidate Substances

As used herein the term “candidate substance” refers to any molecule that is capable of modulating TR2, TR4 or TR2/TR4 heterodimer protein activity or expression. The candidate substance may be a protein or fragment thereof, an antibody or antigen binding fragment thereof, a small molecule, or a nucleic acid molecule.

It may prove to be the case that the most useful pharmacological compounds for identification through application of the screening assays will be compounds that are structurally related to other known agents typically used to treat hemoglobinopathies. The active compounds may include fragments or parts of naturally-occurring compounds or may be only found as active combinations of known compounds which are otherwise inactive. However, prior to testing of such compounds in humans or animal models, it will be necessary to test a variety of candidates to determine which have potential.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds molded of active, but otherwise undesirable compounds.

There are a number of different libraries used for the identification of small molecule modulators including chemical libraries, natural product libraries and combinatorial libraries comprised of random or designed peptides, oligonucleotides or organic molecules. Chemical libraries consist of structural analogs of known compounds or compounds that are identified as hits or leads via natural product screening or from screening against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review see Science 282:63-68 (1998). Combinatorial libraries are composed of large numbers of peptides oligonucleotides or organic compounds as a mixture. They are relatively simple to prepare by traditional automated synthesis methods, PCR cloning or other synthetic methods. Of particular interest will be libraries that include peptide, protein, peptidomimetic, multiparallel synthetic collection, recombinatorial and polypeptide libraries. A review of combinatorial libraries and libraries created therefrom, see Myers Curr. Opin. Biotechnol. 8:701-707 (1997). Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection; Bethesda, Md., NCI's Developmental Therapeutics Program, or the like. A candidate substance identified by the use of various libraries described herein is optimized to modulate activity of TR2, TR4 or TR2/TR4 heterodimer protein through, for example, rational drug design.

It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other inorganic or organic chemical compounds that may be designed through rational drug design starting from known agents that are used in the intervention of hemoglobinopathies.

Antibodies that are specific for TR2, TR4 or the TR2/TR4 heterodimer are also contemplated as modulators of erythroid differentiation. As used herein, the term “antibody” is used in the broadest sense and includes fully assembled antibodies, monoclonal antibodies, polyclonal antibodies, multispecific antibodies (including bispecific antibodies), antibody fragments that can bind an antigen (including, Fab′, F′(ab)₂, Fv, single chain antibodies, diabodies), and recombinant peptides comprising the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity.

Antibodies are “specific for TR2” when it has significantly higher binding affinity for, and consequently is capable of distinguishing, TR2 compared to other unrelated proteins in different families.

Antibodies are “specific for TR4” when it has significantly higher binding affinity for, and consequently is capable of distinguishing, TR4 compared to other unrelated proteins in different families.

Antibodies are “specific for the TR2/TR4 heterodimer” when it has significantly higher binding affinity for, and consequently is capable of distinguishing, the TR2/TR4 heterodimer compared to other unrelated proteins in different families.

The term “derivative” refers to a molecule (e.g., an antibody, a peptide, etc.) that is covalently modified by conjugation to therapeutic or diagnostic agents, labeling (e.g., with radionuclides or various enzymes), covalent polymer attachment such as pegylation (derivatization with polyethylene glycol) and insertion or substitution by chemical synthesis of non-natural amino acids. Derivatives of the invention will retain the binding properties of underivatized molecules of the invention.

Standard techniques are employed to generate polyclonal or monoclonal antibodies directed against TR2, TR4 or the TR2/TR4 heterodimer, and to generate useful antigen-binding fragments thereof or variants thereof. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988).

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay.

In one embodiment, a host animal (including but not limited to, rabbits, mice, rats, guinea pigs, horses, sheep, goats, etc) is immunized by injection with an antigen (e.g., TR2, TR4 or the TR2/TR4 heterodimer). In another embodiment, the antigen is conjugated to an immunogenic carrier known in the art. Various adjuvants known in the art may be used to increase the immunological response, depending on the host species, including but not limited to Freund's (complete and incomplete), mineral gels (e.g., aluminum hydroxide), surface-active substances (e.g., lysolecithin, pluronic polyols, polyanions, peptides, oil emulsions, dinitrophenol, and potentially useful human adjuvants such as Bacille Calmette Guerin).

For preparation of monoclonal antibodies directed toward TR2, TR4 or the TR2/TR4 heterodimer, any technique that provides for the production of antibody molecules by continuous cell lines in culture is contemplated (See e.g., Harlow and Lane, Antibodies: A Laboratory Manual, Cold Spring Harbor Laboratory Press, Cold Spring Harbor, N.Y.). These techniques include, but are not limited to, the hybridoma technique (Köhler and Milstein, Nature 256:495-497, 1975), the trioma technique, the human B-cell hybridoma technique (See e.g., Kozbor et al., Immunol Today, 4:72, 1983), and the EBV-hybridoma technique to produce human monoclonal antibodies (Cole et al., in Monoclonal Antibodies and Cancer Therapy, Alan R. Liss, Inc., pp. 77-96, 1985).

In addition, techniques known in the art the production of single chain antibodies (U.S. Pat. No. 4,946,778 herein incorporated by reference) are contemplated. An additional embodiment of the invention utilizes the techniques described for the construction of Fab expression libraries (Huse et al., Science, 246:1275-1281, 1989) to allow rapid and easy identification of monoclonal Fab fragments with the desired specificity for TR2, TR4 or the TR2/TR4 heterodimer.

In another aspect, techniques known in the art for producing antibody fragments that contain the idiotype (i.e., antigen-binding region) of the antibody molecule are contemplated. Nonlimiting examples of antibody fragments include Fab, Fab′, F(ab′)₂, Fv [variable region], domain antibodies (dAb, containing a VH domain) (Ward et al., Nature 341:544-546, 1989], complementarity determining region (CDR) fragments, single-chain antibodies (scFv, containing VH and VL domains on a single polypeptide chain) (Bird et al., Science 242:423-426, 1988, and Huston et al., Proc. Natl. Acad. Sci. USA 85:5879-5883, 1988, optionally including a polypeptide linker; and optionally multispecific, Gruber et al., J. Immunol. 152: 5368 (1994)), single chain antibody fragments, diabodies (VH and VL domains on a single polypeptide chain that pair with complementary VL and VH domains of another chain) (EP 404,097; WO 93/11161; and Hollinger et al., Proc. Natl. Acad. Sci. USA, 90:6444-6448 (1993)), triabodies, tetrabodies, minibodies (scFv fused to CH3 via a peptide linker (hingeless) or via an IgG hinge) (Olafsen, et al., Protein Eng Des Sel. 2004 April; 17(4):315-23), linear antibodies (tandem Fd segments (VH-CH1-VH-CH1) (Zapata et al., Protein Eng., 8(10):1057-1062 (1995)); chelating recombinant antibodies (crAb, which can bind to two adjacent epitopes on the sane antigen) (Neri et al., J Mol Biol. 246:367-73, 1995), bibodies (bispecific Fab-scFv) or tribodies (trispecific Fab-(scFv)(2)) (Schoonjans et al., J Immunol. 165:7050-57, 2000; Willems et al., J Chromatogr B Analyt Technol Biomed Life Sci. 786:161-76, 2003), intrabodies (Biocca, et al., EMBO J. 9:101-108, 1990; Colby et al., Proc Natl Acad Sci USA. 101:17616-21, 2004) which may also comprise cell signal sequences which retain or direct the antibody intracellularly (Mhashilkar et al, EMBO J 14:1542-51, 1995; Wheeler et al., FASEB J. 17:1733-5, 2003), transbodies (cell-permeable antibodies containing a protein transduction domain (PTD) fused to scFv (Heng et al., Med Hypotheses. 64:1105-8, 2005), nanobodies (approximately 15 kDa variable domain of the heavy chain) (Cortez-Retamozo et al., Cancer Research 64:2853-57, 2004), small modular immunopharmaceuticals (SMIPs) (WO03/041600, U.S. Patent publication 20030133939 and US Patent Publication 20030118592), an antigen-binding-domain immunoglobulin fusion protein, a camelized antibody (in which VH recombines with a constant region that contains hinge, CH1, CH2 and CH3 domains) (Desmyter et al., J. Biol. Chem. 276:26285-90, 2001; Ewert et al., Biochemistry 41:3628-36, 2002; U.S. Patent Publication Nos. 20050136049 and 20050037421), a VHH containing antibody, heavy chain antibodies (HCAbs, homodimers of two heavy chains having the structure H2L2), or variants or derivatives thereof, and polypeptides that contain at least a portion of an immunoglobulin that is sufficient to confer specific antigen binding to the polypeptide, such as a CDR sequence, as long as the antibody retains the desired biological activity.

The term “variant” when used in connection with antibodies refers to a polypeptide sequence of an antibody that contains at least one amino acid substitution, deletion, or insertion in the variable region or the portion equivalent to the variable region, provided that the variant retains the desired binding affinity or biological activity. In addition, the antibodies of the invention may have amino acid modifications in the constant region to modify effector function of the antibody, including half-life or clearance, ADCC and/or CDC activity. Such modifications can enhance pharmacokinetics or enhance the effectiveness of the antibody in treating cancer, for example. See Shields et al., J. Biol. Chem., 276(9):6591-6604 (2001), incorporated by reference herein in its entirety. In the case of IgG1, modifications to the constant region, particularly the hinge or CH2 region, may increase or decrease effector function, including ADCC and/or CDC activity. In other embodiments, an IgG2 constant region is modified to decrease antibody-antigen aggregate formation. In the case of IgG4, modifications to the constant region, particularly the hinge region, may reduce the formation of half-antibodies.

Methods for making bispecific or other multispecific antibodies are known in the art and include chemical cross-linking, use of leucine zippers [Kostelny et al., J. Immunol. 148:1547-1553, 1992]; diabody technology [Hollinger et al., Proc. Natl. Acad. Sci. USA 90:6444-48, 1993]; scFv dimers [Gruber et al., J. Immunol. 152: 5368, 1994], linear antibodies [Zapata et al., Protein Eng. 8:1057-62, 1995]; and chelating recombinant antibodies [Neri et al., J Mol Biol. 246:367-73, 1995].

Antibodies generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference, are also contemplated.

Screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

An “isolated” antibody refers to an antibody, as that term is defined herein, that has been identified and separated from a component of its natural environment. Contaminant components of its natural environment are materials that would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody, and most preferably more than 99% by weight, (2) to a degree sufficient to obtain at least 15 residues of N-terminal or internal amino acid sequence, or (3) to homogeneity by SDS-PAGE under reducing or nonreducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

Inhibitory oligonucleotides or polynucleotides (including pharmaceutically acceptable salts thereof) are also contemplated as modulators of erythroid differentiation. Nonlimiting examples include antisense oligonucleotides [Eckstein, Antisense Nucleic Acid Drug Dev., 10: 117-121 (2000); Crooke, Methods Enzymol., 313: 3-45 (2000); Guvakova et al., J. Biol. Chem., 270: 2620-2627 (1995); Manoharan, Biochim. Biophys. Acta, 1489: 117-130 (1999); Baker et al., J. Biol. Chem., 272: 11994-12000 (1997); Kurreck, Eur. J. Biochem., 270: 1628-1644 (2003); Sierakowska et al., Proc. Natl. Acad. Sci. USA, 93: 12840-12844 (1996); Marwick, J. Am. Med. Assoc. 280: 871 (1998); Tomita and Morishita, Curr. Pharm. Des., 10: 797-803 (2004); Gleave and Monia, Nat. Rev. Cancer, 5: 468-479 (2005) and Patil, AAPS J. 7: E61-E77 (2005], triplex oligonucleotides [Francois et al., Nucleic Acids Res., 16: 11431-11440 (1988) and Moser and Dervan, Science, 238: 645-650 (1987)], ribozymes/deoxyribozymes(DNAzymes) [Kruger et al., Tetrahymena. Cell, 31: 147-157 (1982); Uhlenbeck, Nature, 328: 596-600 (1987); Sigurdsson and Eckstein, Trends Biotechnol., 13 286-289 (1995); Kumar et al., Gene Ther., 12: 1486-1493 (2005); Breaker and Joyce, Chem. Biol., 1: 223-229 (1994); Khachigian, Curr. Pharm. Biotechnol., 5: 337-339 (2004); Khachigian, Biochem. Pharmacol., 68: 1023-1025 (2004) and Trulzsch and Wood, J. Neurochem., 88: 257-265 (2004)], small-interfering RNAs/RNAi [U.S. Pat. Nos. 6,278,039, 5,723,750 and 5,244,805; Fire et al., Nature, 391: 806-811 (1998); Montgomery et al., Proc. Natl. Acad. Sci. U.S.A., 95: 15502-15507 (1998); Cullen, Nat. Immunol., 3: 597-599 (2002); Hannon, Nature, 418: 244-251 (2002); Bernstein et al., Nature, 409: 363-366 (2001); Nykanen et al., Cell, 107: 309-321 (2001); Gilmore et al., J. Drug Target., 12: 315-340 (2004); Reynolds et al., Nat. Biotechnol., 22: 326-330 (2004); Soutschek et al., Nature, 432173-178 (2004); Ralph et al., Nat. Med., 11: 429-433 (2005); Xia et al., Nat. Med., 10816-820 (2004) and Miller et al., Nucleic Acids Res., 32: 661-668 (2004)], short hairpin RNA [Hannon et al., Nature, 431:371-378, 2004; Brummelkamp et al., Science 296, 550-553, 2000; Paddison et al., Genes Dev. 16, 948-958 (2002); Dirac, et al., J. Biol. Chem. 278:11731-11734, 2003; Michiels et al., Nat. Biotechnol. 20:1154-1157, 2002; Stegmeie et al., Proc. Natl. Acad. Sci. USA 102:13212-13217, 2005; Khvorova et al., Cell, 115:209-216 (2003)], aptamers [Ellington and Szostak, Nature, 346: 818-822 (1990); Doudna et al., Proc. Natl. Acad. Sci. U.S.A., 92: 2355-2359 (1995); Tuerk and Gold, Science, 249: 505-510 (1990); White et al., Mol. Ther., 4: 567-573 (2001); Rusconi et al., Nature, 419: 90-94 (2002); Nimjee et al., Mol. Ther., 14: 408-415 (2006); Gragoudas et al., N. Engl. J. Med., 351: 3805-2816 (2004); Vinores, Curr. Opin. Mol. Ther., 5673-679 (2003) and Kourlas and Schiller et al., Clin. Ther., 28 36-44 (2006)], ribozymes [Rossi, 1994, Current Biology 4:469-471] or decoy oligonucleotides [Morishita et al., Proc. Natl. Acad. Sci. U.S.A., 92: 5855-5859 (1995); Alexander et al., J. Am. Med. Assoc., 294: 2446-2454 (2005); Mann and Dzau, J. Clin. Invest., 106: 1071-1075 (2000) and Nimjee et al., Annu. Rev. Med., 56: 555-583 (2005). The foregoing documents are hereby incorporated by reference in their entirety herein. Commercial providers such as Ambion Inc. (Austin, Tex.), Darmacon Inc. (Lafayette, Colo.), InvivoGen (San Diego, Calif.), and Molecular Research Laboratories, LLC (Herndon, Va.) generate custom siRNA molecules. In addition, commercial kits are available to produce custom siRNA molecules, such as SILENCER™ siRNA Construction Kit (Ambion Inc., Austin, Tex.) or psiRNA System (InvivoGen, San Diego, Calif.).

Inhibitory oligonucleotides can be administered directly or delivered to cells by transformation or transfection via a vector, including viral vectors or plasmids, into which has been placed DNA encoding the inhibitory oligonucleotide with the appropriate regulatory sequences, including a promoter, to result in expression of the inhibitory oligonucleotide in the desired cell. Known methods include standard transient transfection, stable transfection and delivery using viruses ranging from retroviruses to adenoviruses. Delivery of nucleic acid inhibitors by replicating or replication-deficient vectors is contemplated. Expression can also be driven by either constitutive or inducible promoter systems (Paddison et al., Methods Mol. Biol. 265:85-100, 2004). In other embodiments, expression may be under the control of tissue or development-specific promoters.

In Vitro Assays.

In one embodiment, the invention provides in vitro binding assays. In such binding assays, the TR2, TR4 or TR2/TR4 heterodimer protein or a fragment thereof is either free in solution, fixed to a support, or expressed in or on the surface of a cell. Either the polypeptide or the candidate substance is optionally labeled, thereby permitting determination of binding.

Such assays are highly amenable to automation and high throughput. High throughput screening of compounds is described in WO 84/03564. Large numbers of candidate substances are synthesized on a solid substrate, such as plastic pins, 96-well plate, beads or some other solid surface. Candidate substances are reacted with TR2, TR4 or TR2/TR4 heterodimer protein and washed. Bound polypeptide is detected by various methods. Combinatorial methods for generating suitable test compounds are specifically contemplated.

Purified TR2, TR4 or TR2/TR4 heterodimer protein or a candidate substance is coated directly onto plates for use in the aforementioned drug screening techniques. However, non-neutralizing antibodies to the TR2, TR4 or TR2/TR4 heterodimer may optionally be used to immobilize the polypeptide to a solid phase. Also, fusion proteins containing a reactive region may be used to link the TR2, TR4 or TR2/TR4 heterodimer protein active region to a solid phase.

Other forms of in vitro assays include those in which functional readouts are taken. For example cells in which a wild-type or mutant TR2, TR4 or TR2/TR4 heterodimer protein polypeptide is expressed can be treated with a candidate substance. In such assays, the candidate substance is formulated appropriately, given its biochemical nature, and contacted with the cell. Depending on the assay, culture may be required. The cell is then examined by virtue of a number of different physiologic assays. Alternatively, molecular analysis is performed in which the cells characteristics are examined. This may involve assays such as those for protein expression, enzyme function, substrate utilization, mRNA expression (including differential display of whole cell or polyA RNA) and others.

In one aspect, a high throughput assay to screen for compounds that affect TR2, TR4 or the TR2/TR4 heterodimer activity may be based on the method described by Farrelly et al. Analytical Biochemistry 293:269-276 (2001).

In Vivo Assays.

The use of various animal models of hemoglobinopathies is contemplated. Exemplary animal models of hemoglobinopathies include those models disclosed in Yang et al., Proc. Natl. Acad. Sci. USA., 92:11608-11612, 1995; Suzuki et al., Biochem, Biophys. Res. Commum., 295:869-876, 2002; Lewis et al., Blood, 91:2152-2156, 1998; Rivella et al., Blood, 8:2932-2939, 2003; Fabry et al., Ann. N.Y. Acad. Sci., 565:379, 1989; Kurantsin-Mills et al., Prog. Clin. Biol. Res., 240:313-327, 1987; Nagel, R., N. Engl. J. Med., 339:194-195, 1998; and Nagel et al., Br. J. Haematol., 112:19-25, 2001, the disclosures of which are incorporated herein by reference in their entireties, and other animal models known in the art. Animal models afford the skilled artisan an opportunity to examine the function of TR2, TR4 or TR2/TR4 heterodimer protein and its modulators in a whole animal system where it is normally expressed. Thus, candidate substances can be identified in initial in vitro screens as discussed above and then further tested in whole animal screens. In this manner, one can achieve test results with the candidate substances that will be highly predictive of efficacy in humans and other mammals, and helpful in identifying potential therapies.

Treatment of animals with a candidate substance will involve the administration of the candidate substance, in an appropriate form, to the animal. Administration will be by any route that can be utilized for clinical or non-clinical purposes, including but not limited to oral, nasal, buccal, rectal, vaginal, topical, intradermal, subcutaneous, intramuscular, intraperitoneal or intravenous injection.

Determining the effectiveness of a compound in vivo may involve a variety of different criteria. Such criteria include, but are not limited to, erythroid cell survival, stimulation or inhibition of globin expression, stimulation or inhibition of TR2/TR4 heterodimer formation, etc.

Candidate substances identified from such screening assays are then formulated into pharmaceutical compositions for use as therapeutic (or prophylactic) agents. Such substances may be orally available small molecule compounds. In an alternative embodiment, such compositions are selected from among small molecules, antisense molecules, siRNA, therapeutic antibodies and the like.

Any of the therapeutic agents identified by the above screens may be administered alone as therapeutic agents or alternatively may be administered in combination with other known treatments for hemoglobinopathies including hydroxyurea, cytidine analog 5-azacytidine (5-azaC), erythropoietin, erythropoietin stimulators, blood transfusion and bone marrow transplants.

In order to carry out the methods of the present invention, in one aspect, the skilled artisan employs a wide variety of tools for use in research which employ TR2, TR4 or the TR2/TR4 heterodimer. For example, purified TR2, TR4 or the TR2/TR4 heterodimer genes or proteins, recombinant cells containing additional copies of such gene(s), antibodies to TR2, TR4 or the TR2/TR4 heterodimer, and transgenic animals, such as mice created to have non-functional forms of the gene (knock-out or knock-down) or recombinant mice having additional copies of the gene(s) are contemplated. Recombinant techniques for the purification TR2, TR4 or the TR2/TR4 heterodimer genes, preparation of recombinant cells that express TR2, TR4 or the TR2/TR4 heterodimer proteins, preparations of isolated recombinant such proteins are routine well known to those of skill in the art. See for example, Molecular Cloning: A Laboratory Manual, Sambrook et al., Cold Spring Harbor Press, (2001). Any recombinant cell can be used. The cell may be an erythroid cell, or any other mammalian cell that can be transfected with an TR2, TR4 or the TR2/TR4 heterodimer-encoding nucleic acid.

Gene Therapy

Delivery of a modulator of TR2, TR4 or TR2/TR4 heterodimer activity to appropriate cells can be effected via gene therapy ex vivo, in situ, or in vivo by use of any suitable approach known in the art. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187.

For in vivo therapy, a nucleic acid encoding the desired TR2, TR4 or TR2/TR4 heterodimer activity antagonist or TR2, TR4 or TR2/TR4 heterodimer expression inhibitor, either alone or in conjunction with a vector, liposome, or precipitate may be injected directly into the subject. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187. For review of gene marking and gene therapy protocols, see Anderson 1992. See also WO 93/25673 and the references cited therein. For additional reviews of gene therapy technology, see Gardlik et al., Med. Sci. Mon, 11(4):110-121, (2005); Gao et al., AAPS J., 9:E91-104, (2005) Friedmann, Science, 244: 1275-1281 (1989); Anderson, Nature, supplement to vol. 392, no 6679, pp. 25-30 (1998); Verma, Scientific American: 68-84 (1990); and Miller, Nature, 357: 455460 (1992) and the references cited therein. The disclosure of each of the foregoing documents is incorporated herein by reference in their entireties.

Identified Candidate Agents in Pharmaceutical Compositions and Methods of Treatment

In one embodiment, the methods of the present invention are used to treat a disease or disorder in a subject in need. The subject to be treated is any vertebrate, and preferably is human.

In some aspects, the methods are used to treat or pretreat a subject having or at risk for having a hemoglobinopathy. Such hemoglobinopathies include any disorder associated with an alteration in the amount, structural integrity, or function of adult hemoglobin. In one aspect, the adult hemoglobin is adult β globin. Hemoglobinopathies specifically include, but are not limited to, β thalassemia including β^{0-(major)} and β^{+-(minor)} thalassemia, and sickle cell disease including sickle cell anemia and sickle 0 thalassemia.

Also provided are methods of treating aberrant globin expression in a subject comprising contacting a TR2/TR4 heterodimer with an inhibitor that prevents binding of the TR2/TR4 heterodimer to a γ-globin promoter sequence. In one aspect, the aberrant globin expression is associated with expression of an abnormal 13 globin gene product

In some embodiments, the methods comprise administering to a patient in need an effective amount of a compound that increases γ globin production in a cell or population of cells.

In one aspect, the compound inhibits the activity or expression (or the formation of) of TR2, TR4 or the TR2/TR4 heterodimer. The compound may be administered alone, in a pharmaceutically acceptable formulation, or in combination with one or more therapeutic agents. In some embodiments, the second therapeutic agent additively or synergistically increases γ globin production. Such therapeutic agents include, but are not limited to, hydroxyurea, butyrate analogs, and 5-azacytidine. (See, e.g., U.S. Pat. No. 5,569,675; U.S. Pat. No. 5,700,640; U.S. Pat. No. 6,231,880; International Publication No. WO 93/18761; and International Publication No. WO 97/12855.) In other embodiments, the therapeutic agent(s) provide a therapeutic benefit to patients in need by a mechanism distinct from y globin induction, e.g., such agents may include, but are not limited to, Gardos channel inhibitors (see, e.g., U.S. Pat. No. 6,218,122; U.S. Pat. No. 6,331,564; International Publication No. WO 99/24034; and International Publication No. WO 96/08242), which inhibit dehydration of red blood cells; and/or hematopoietic growth factors such as erythropoietin and analogs thereof (Epoetin alfa, Epoetin beta, Epoetin iota, Epoetin delta, Epoetin omega, Epoetin zeta, darbepoetin alfa), mimetic peptides, mimetic antibodies, hypoxia-inducible factor (HIF) inhibitors (U.S. Patent Publication No. 2005/0020487, the disclosure of which is incorporated herein by reference in its entirety), GM-CSF, and IL-3.

In another embodiment, the methods of the invention are used to treat cells ex vivo to induce γ-globin production. The cells are then transfused into a patient in need of treatment. In a particular embodiment, the cells are bone marrow cells. The bone marrow may be derived from the patient (autologous) or from a matched donor (allogenic). Such methods can be used in conjunction with current blood transfusions, e.g., for treatment of sickle cell disease.

In some aspects, the methods are used to treat patients with sickle cell disease who do not respond to treatment with other therapeutic agents, e.g., hydroxyurea, etc. In other aspects, the methods are used to augment treatment with another therapeutic agent. The use of the current method in conjunction with a second therapeutic may be required because, e.g., the patient may only partially respond to current therapy. The use of the current methods in conjunction with a second therapeutic agent may facilitate reducing the required dosage of the second agent to avoid pharmacokinetic or toxicity associated with the agent. For example, current therapies in hemoglobinopathies are limited by factors including poor pharmacokinetics, e.g., butyrate requires extremely high doses for therapeutic efficacy (Dover et al. (1994) Blood 84(1):339-343); and dose-limiting toxicity associated with, e.g. butyrate, 5-azacytidine, and hydroxyurea. (See, e.g., Blau et al. (1993) Blood 81:529-537; Ley and Nienhuis (1985) Annu Rev Med 36:485-498; DeSimone et al. (2002) Blood 99:3905-3908; and Charache et al. (1995) N Engl J Med 332:1317-1322.)

Continuous formation and destruction of irreversibly sickled cells contributes significantly to the severe hemolytic anemia that occurs in patients with sickle cell disease. The anemia of sickle cell can be even more severe if erythropoiesis is suppressed. For example, folic acid and vitamin B₁₂ are required for proper cell division; and deficiency in these nutrients leads to enlarged blood cells (megaloblastic cells), which are destroyed in the marrow, thus causing anemia due to ineffective erythropoiesis. Further, a deficiency in iron, which is necessary for functional heme production, further aggravates the anemia. Therefore, in yet another embodiment, the compound is administered with a second agent selected from the group consisting of folic acid, vitamin B₁₂, and an iron supplement, e.g., ferrous gluconate.

Pharmaceutical Compositions and Routes of Administration

The compounds identified in the methods of the present invention can be delivered directly or in pharmaceutical compositions along with suitable carriers or excipients, as is well known in the art. Present methods of treatment can comprise administration of an effective amount of a compound of the invention to a subject having or at risk for hemoglobinopathies including β thalassemia major, β thalassemia minor, sickle cell disease, etc. In a preferred embodiment, the subject is a primate, and in a most preferred embodiment, the subject is a human.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, Ed. (2000) Remington's Pharmaceutical Sciences, supra; and Hardman, Limbird, and Gilman, Eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methan and ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the compounds of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the invention, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or medicament that will elicit the biological or medical response of a tissue, system, animal or human that is being sought by the researcher, veterinarian, medical doctor or other clinician, e.g., reduced TR2, TR4, TR2/TR4 heterodimer activity, reduced SCD crises, etc.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition. Suitable conditions indicated on the label may include treatment of hemoglobinopathies.

Other aspects and advantages of the present invention will be understood upon consideration of the following illustrative examples, which are not intended to be limiting in any way.

### EXAMPLES

### Example 1

### Materials and Methods

Plasmids. For transient expression in cell culture, the mouse TR2 and TR4 cDNAs were appended to Flag-tags at their amino-termini (Tanabe et al., 2002), and then cloned between the Nhe I and Not I sites of a CMV promoter-driven expression vector pEGFP-N3 (Clontech), replacing the eGFP gene. cDNAs encoding TR2 and TR4 mutants were generated by PCR-directed mutagenesis, and cloned into the same vector. For transgenic expression, the Flag-tagged mouse TR2 or TR4 cDNAs, the dominant negative TR4 mutant, and eGFP (Sma I-Not I fragment) from the pEGFP-N3 plasmid were ligated to the Kpn I-Not I fragment from IE3.9int-LacZ (GATA1-HRD, (Onodera et al., 1997)) followed by the SV40 polyadenylation signal.

Mice. The Tr2 and Tr4 null mutant mice were described previously (Shyr et al., 2002; Collins et al., 2004). For generation of transgenic mice expressing wild-type TR2 or TR4, or the dominant negative TR4 mutant, the expression DNAs were separated from the plasmid backbones by restriction enzyme digestion, agarose gel electrophoresis, and electroelution. The constructs were injected into fertilized mouse oocytes (CD1; Charles River Breeding Laboratory) that were then transferred to foster dams as described (Nagy et al., 2003). Tail DNA from founder offspring was initially analyzed for the presence of TR2 or TR4 cDNA, or the eGFP gene, by PCR. Transgenic founder mice were bred to wild type CD1 mice and F1 offspring were analyzed for the presence of the transgenes by Southern blots of DNA prepared from tail snips. Transgenic mouse lines bearing the wild-type human β-globin YAC (line 264; Tanimoto et al., 1999) and the mutant YAC transgenes Bepsi (line 588; Tanimoto et al., 2002) and mutDR (line 74; Omori et al., 2005) were described previously.

Semi-quantitative RT-PCR Analysis of Mouse and Human Globin mRNAs. Total RNA from transgenic lines was extracted from the yolk sac, fetal liver, or adult spleen using Trizol (Invitrogen) or Isogen (Nippon Gene). Prior to RNA extraction from adult spleen samples, anemia was induced by intraperitoneal injection of three doses (40 μg/g body weight) of 1-acetyl-2-phenylhydrazine (Sigma) over 24 hours, and then the spleen was collected 4 days later. First-strand cDNA was synthesized with Superscript II using 1 μg of total RNA in a 20 μl reaction volume. PCR was performed using 1 μl of cDNA in a 10 μl reaction volume final) containing 20 mM Tris-HCl (pH8.4), 50 mM KCl, 2.5 mM MgCl2, 200 μM each dNTP, 0.25 units of Taq DNA polymerase (Invitrogen), 0.5 μCi of [α32P]-dCTP (ICN), and each gene-specific primer (3.75 pmole for mouse globin or 3 pmole for human globin) with temperature cycles of denaturation at 94° C. for 30 sec, annealing at 57° C. (for mouse globin) or 58° C. (for human globin) for 30 sec, and extension at 72° C. for 1 min. Optimal PCR cycle numbers within the exponential amplification range were experimentally determined for each primer set and each tissue. PCR reactions were electrophoresed on 8% polyacrylamide gels, dried, and quantified on a PhosphoImager (Molecular Dynamics). The following primers were used for PCR of mouse globin cDNAs: εy: 5′-ACCCTCATCAATGGCCTGTGGA-3′ (SEQ ID NO: 1) and 5′-CATGGGCTTTGACCCTTGGG-3′ (SEQ ID NO: 2) (166 bp amplicon); βh1: 5′-ATCATGGGAAACCCCCGGA-3′ (SEQ ID NO: 3) and 5′-GGGTGAATTCCTTGGCAAAATGAGT-3′ (211 bp amplicon) (SEQ ID NO: 4); β^{major}: 5′ TCTGCTATCATGGGTAATGCCAAA-3′ (SEQ ID NO: 5) and 5′-GAAGGCAGCCTGTGCAGCG-3′ (237 bp amplicon) (SEQ ID NO: 6); mouse a: 5′-GCTGCCTGGGGGAAGATTGG-3′ (SEQ ID NO: 7) and 5′-GGGTGAAATCGGCAGGGTGG-3′ (322 bp amplicon) (SEQ ID NO: 8). All these primer sets were designed to span introns in order to amplify only cDNAs. The PCR primers for human globin cDNAs were previously described (Tanimoto et al., 1999). For human globin RT-PCR analysis using yolk sac cDNAs, the amplification products for ε (12-13 cycles), γ (12 cycles), or β (12 cycles) PCR were quantified and normalized to the co-amplified mouse α-globin amplicon. For fetal liver analysis, ε (18 cycles) and γ (16 cycles) were normalized to mouse α, amplified separately (10 cycles), while 3 (12 cycles) was normalized to co-amplified mouse α. For adult spleen analysis, γ (22 cycles) was normalized to mouse α, amplified separately (10 cycles), while 3 (10 cycles) was normalized to co-amplified mouse α. To determine the molar ratio of Gγ to Aγ mRNAs, both Gγ and Aγ cDNAs were co-amplified with common γ primers, and then digested with Pst I (digests only the Aγ amplicon) to produce distinct fragments on gel electrophoresis as described previously (Omori et al., 2005). The Gγ/Aγ molar ratio was calculated based on the numbers of C nucleotides in each fragment. The relative abundance (molar ratio) of β-type globin RNAs normalized to mouse α-globin mRNA was calculated associng to the following equation:

$$Molar ratio ( \% ) = \frac{R β}{R α} ⨯ \frac{C α}{C β} ⨯ \frac{{( 1 + E α )}^{N α}}{{( 1 + E β )}^{N β}} ⨯ 100$$

where Rβ or Rα is the radioactivity of PCR product for β-type or mouse α-globin measured by PhosphoImager; Cβ or Cα is the number of C nucleotides incorporated by PCR in each amplicon for β-type of α-globin; Eβ or Eα amplification efficiency of a primer set for β-type or α globin; Nβ or Nα is the number of PCR cycles for β-type or α-globin. The amplification efficiency for each primer set was experimentally determined for each tissue by plotting radioactivity of PCR products against cucle numbers over a 6-8 cycle range.

Real-time PCR Analysis for Quantifying TR2, TR4, and GATA-1 cDNA. Real-time PCR analysis was performed with 0.1 μl of 14.5 dpc fetal liver cDNA prepared as described above in a 25 μl reaction using the ABI Prism 7000 Sequence Detection System and SYBR Green PCR Master Mix (Applied Biosystems). The following primers for each cDNA were used: endogenous TR2: 5′-GGATATTTTGACCATTCGATCATG-3′ (SEQ ID NO: 9) and 5′-CCAGTCTGCTGCTCTGTAACAATCT-3′ (SEQ ID NO: 10); transgene-derived TR2: 5′-CATCAACAAGCCCAGGTTCAA-3′ (SEQ ID NO: 11) and 5′-CTCACCCATCTGTTGGTCGA-3′ (SEQ ID NO: 12); endogenous TR4: 5′-CCGGAATCTCCAGGGATGA-3′ (SEQ ID NO: 13) and 5′-TCTTTTGTCCTGTCTGCTGGTCT-3′ (SEQ ID NO: 14); transgene-derived TR4: 5′-CATCAACAAGCCCAGGTTCAA-3′ (SEQ ID NO: 15) and 5′-AGGTGAGGCTACCGCAGAGT-3′ (SEQ ID NO: 16); GATA-1: 5′-CTGCATCAACAAGCCCAGG-3′ (SEQ ID NO: 17) and 5′-GGGCCCCTAGACCAGGAAA-3′ (SEQ ID NO: 18). All these primer sets were designed to span introns in order to amplify only cDNAs. The abundance of each cDNA was determined based on its Ct value and an experimentally determined amplification efficiency for each primer set, and then normalized to the abundance of GATA-1 cDNA (internal control).

Transient Embryonic Anemia. 10.5 to 14.5 dpc TR4 transgenic embryos and their wild type littermates were separated from the uterus together with intact yolk sacs and placentas, and then rinsed with phosphatebuffered saline (PBS) several times to remove maternal blood. The macroscopic appearance of the embryos was determined immediately after rupturing the yolk sacs to minimize blood loss. To count blood cells, the 10.5 to 12.5 dpc embryos rinsed with PBS were placed in containers with 0.5 ml PBS and then detached from the yolk sac and placenta. Blood was collected from the embryos by piercing and compressing the heart with forceps. The number of erythrocytes in the embryonic blood thus collected was determined with a hemocytometer.

Cell Culture and Transient Transfection. The mouse erythroleukemia cell line MEL and human embryonic kidney cell line 293T were cultured in Dulbecco's modified Eagles medium supplemented with 10% fetal calf serum. For transfection, 2×10⁶ 293T cells were plated in a 10 cm dish the day before transfection. 20 μg of expression plasmid was mixed with 40 μl of lipofectamine 2000 (Invitrogen) and then added to each dish according to the manufacturer's instruction. Forty-eight hours after transfection, cells were harvested and nuclear extracts were prepared.

Electrophoretic Gel Mobility Shift Assay (EMSA). Nuclear extracts preparation, binding reactions, and electrophoresis were performed as described previously (Tanimoto et al., 2000). The following blunt ended oligonucleotides were used as probes or competitors in EMSA (only sense strands are shown): ε distal DR, 5′-CCCTGAGGACACAGGTCAGCCTTG-3′ (SEQ ID NO: 19); ε proximal DR, 5′-CAGCCTTGACCAATGACTTTTA-3′ (SEQ ID NO: 20); γ DR, 5′-GCCTTGCCTTGACCAATAGCCTTGACAA-3′ (SEQ ID NO: 21); β, 5′-TAGGGTTGGCCAATCTACTCCC-3′ (SEQ ID NO: 22); εy distal DR, 5′-CCATGAGGACCACGGGTCAGGCTGA-3′ (SEQ ID NO: 23); εy proximal DR, 5′-TCAGGCTGACCAATGGCTTCAAA-3′ (SEQ ID NO: 24); βh1 DR, 5′-CCCAGACTCTCTTGACCAATAGCCTCAGAGTCCT-3′ (SEQ ID NO: 25); β^{major}, 5′-TGGTAAGGGCCAATCTGCTCACA-3′ (SEQ ID NO: 26).

To determine the affinity of each competitor by EMSA, the dissociation constant (Kd=0.56 nM) for the ³²P-labeled ε distal DR probe was initially determined by saturation binding experiments using nuclear extracts from MEL cells. Free and bound ³²P-labeled probe was quantified on a PhosphoImager. The competitive binding experiments were then performed using 1.1 nM ³²P-labeled ε distal DR probe and 70 pM to 2 μM of each competitor double-strand oligonucleotide to determine the 50% inhibitory concentration (IC₅₀). The equilibrium dissociation constant (Ki) for each competitor was then determined by the following equation (Cheng and Prusoff, 1973):

$${K}_{i} = \frac{{IC}_{50}}{1 + \frac{[ labeled probe ]}{{K}_{d}}}$$

Antibodies. Rabbit antisera against TR2 and TR4 were generated by fusing cDNA fragments [for the amino terminal regions of mouse TR2 (Leu³⁵-Leu¹⁰⁰) and TR4 (Ala⁴³-Tyr¹¹⁶)] that were amplified by PCR into the PshAI and HindIII sites of pET-42a (Novagen), and then expressed as GSTfusion proteins in E. coli BL21-CodonPlus (DE3)-RIL (Stratagene). The fusion proteins were affinity-purified with Glutathione Sepharose 4B (Amersham) according to manufacturers' instructions, and then used as antigen for the preparation of rabbit antisera (Cocalico Biologicals, Inc.). For Western blotting with the anti-TR2 or -TR4 antisera, horseradish peroxidase-conjugated anti-rabbit IgG was used as a secondary antibody for detection using the ECL system (Amersham).

### Example 2

### Analysis of Tr2 and Tr4 Null Mutant Mice

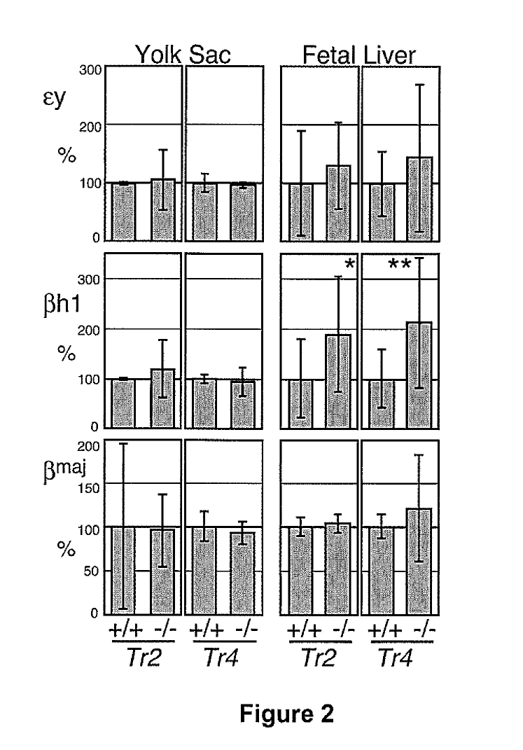

In order to investigate the in vivo roles of TR2 and TR4 in β-type globin gene regulation, the expression of the mouse globin genes in Tr2 or Tr4 null mutant mice (Shyr et al., 2002; Collins et al., 2004) was analyzed. The expression level of the embryonic εy-, βh1-, and adult β^{major} globin genes in 10.5 dpc yolk sac, 13.5 dpc fetal liver, and adult spleen (from acetylphenylhydrazine-induced anemic animals) was determined by semi-quantitative RT-PCR, and normalized to endogenous α-globin mRNA (FIG. 2).

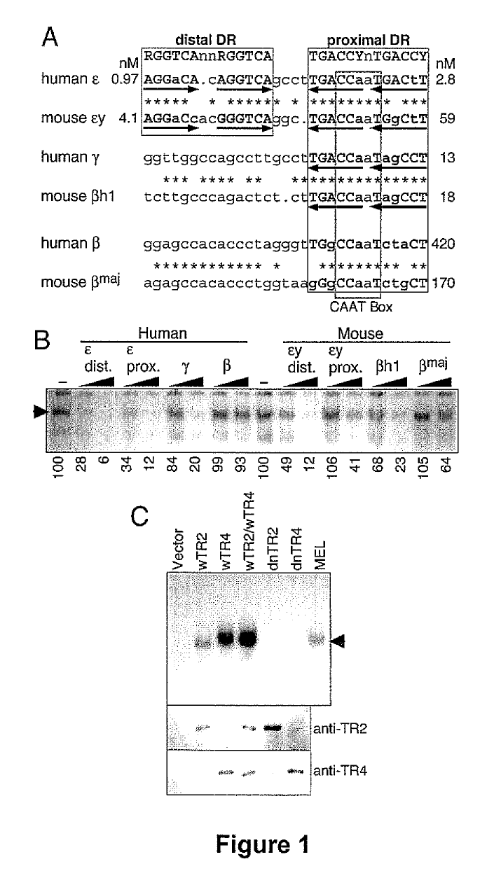

In the yolk sac, there was no significant difference in the expression of any of the globin genes between the homozygous null Tr2 or Tr4 mutant embryos and their wild-type littermates. In the fetal liver, the βh1 gene was induced approximately 2-fold in both Tr2 or Tr4 homozygous null mutant fetuses as compared to their wild-type littermates, but expression of the embryonic εy or adult β^{major} genes was unaffected. In the adult spleen, there was no significant difference in expression of any of the globin genes between the Tr2 or Tr4 null mutants and wildtype mice. These results suggest that TR2 and TR4 play a role in the repression of βh1 in the fetal liver, consistent with the hypothesis that TR2 and TR4 are central components of the DRED complex that represses embryonic and fetal DR-regulated β-type globin genes. However, the results did not provide evidence regarding their roles in the possible regulation of the murine εy globin gene. Because TR2 or TR4 can bind to DR elements as homodimers (albeit with lower affinity than the TR2/TR4 heterodimer, FIG. 1), TR2 and TR4 may be functionally redundant in regulating the embryonic and fetal β-type globin genes, and thus ablation of one gene may be functionally compensated by the other. To examine this possibility, TR2 and TR4 mutants were interbred to generate compound mutants in which to analyze β-globin gene expression

TR2/TR4 mutant loss-of-function effects on human β-type globin transcription were assessed by breeding to a wild-type human β-globin YAC transgenic line (Tb^{βYAC}, Tanimoto et al., 2000). The total amount of transgene-derived human β-type globin mRNAs in animals bearing this “YAC was only about 10% of mouse endogenous α-globin transcript. ε- and γ-globin silencing was significantly delayed (9- or 3.6-fold increased ε- and γ-globin expression in 14.5 d.p.c. fetal livers, respectively) in compound TR2/TR4 homozygous null mutant fetuses as compared to wild type, whereas expression of the human adult β-globin gene was unaffected. In the fetal livers of the Tr2^{-/-}:/Tr4^{-/+} or the Tr2^{-/+}:/Tr4^{-/-} embryos, expression of the ε- and γ-globin genes was induced to levels lying between those of wild-type and compound:homozygous null mutant fetuses. These data indicate that TR2 and TR4 play key roles in repression of the ε- and γ-globin genes, and that TR2 and TR4 are genetically partially redundant. Furthermore, the data suggest that TR4 plays a more prominent role than does TR2 in vivo, as the Tr2^{-/+}:Tr4^{-/-} mutants displayed a more sever phenotype than did the Tr2^{-/-}:/Tr4^{-/+} mutants.

To determine whether these changes in mRNA accumulation were due to altered transcriptional activity, primary RNA transcripts recovered from fetal liver samples of human β-type globin genes were quantified by RT-PCR using primer sets spanning exon-intron junctions (one primer for an exon sequence, and the other for an adjacent intron sequence). The abundance of β-, γ-, or ε-globin primary transcripts in the 14.5/15.5 d.p.c. fetal livers of wild-type mice was about 0.5, 0.2, or 0.2% of the corresponding mRNA abundance, respectively. In the fetal livers of the Tr2^{-/-}:/Tr4^{-/-} mutant fetuses, ε and γ primary transcript levels were elevated 2.9- or 2.4-fold, respectively, compared to wild-type. These data show that TR2 and TR4 exert repressive effects on ε- and γ-gene transcription in definitive erythrois cells of the fetal liver, and thus that the effects of TR2/TR4 are transcriptional and are not due to, for example, altered longevity of primitive erythroid cells or erythroid cell-specific mRNAs.

Discussion:

During embryonic development, primitive erythrocytes produced in the murine yolk sac predominantly express the embryonic εy- and βh1-globins. At around 12.5 dpc when definitive erythropoiesis ensues in the fetal liver, the εy and βh1 genes are gradually silenced with concomitant activation of the two adult (β^{major}- and β^{minor}-globin) genes whose expression continue after birth as the site of erythropoiesis shifts to the bone marrow (Whitelaw et al., 1990). The promoters of the mouse embryonic globin εy and βh1 genes contain either two (εy) or one (βh1) DR elements, and the sequences surrounding those elements are homologous to the equivalent regions of their human orthologues, the embryonic ε- and fetal γ-globin genes, respectively (FIG. 1A).

In order to test this hypothesis that DRED regulates both mouse and human globin genes, electrophoretic gel mobility shift assay (EMSA) competitive binding experiments were performed to determine the affinity of DRED for each DR element in the human and mouse globin gene promoters, using nuclear extracts from the mouse erythroleukemia cell line, MEL, as the source of DRED. A ³²P-labeled probe from the ε-globin distal promoter DR element that conforms best with the consensus binding site for the nuclear receptors of all of the human and mouse β-type globin gene promoter sequences was used; an assortment of unlabeled oligonucleotides corresponding to DR elements of human and mouse β-type globin gene promoters were used as competitors. From these analyses, equilibrium dissociation constants (Kᵢ) representing the affinities of competitor oligonucleotides for DRED were determined.

The data indicated that the Kᵢ value of the human ε distal DR element was 0.97 nM, the highest affinity of all the competitors tested, and the affinity of the human ε proximal DR element (Kᵢ=2.8 nM) was slightly lower than its distal counterpart. The affinity of these sites is similar to the known functional binding sites for TR2 or TR4 (Lee et al., 2002). The affinity of the γ-globin promoter DR element (Kᵢ=13 nM) was slightly lower than the known TR2 or TR4 binding sites, but as high as some of the functional binding sites for RXR (Medin et al., 1994) or HNF-4 (Jiang et al., 1997), other members of the same subfamily of nuclear receptors (Laudet, 1997). The affinity of the human β-globin promoter sequence (Kᵢ=420 nM) was too low to generate functional association. The mouse globin DR sites were of generally lower affinity than the equivalent human gene binding sites. The affinities of the εy distal DR (Kᵢ=4.1 nM) and βh1 DR (Kᵢ=18 nM) elements were sufficiently avid to be functional binding sites, but the affinity of the εy proximal DR element (Kᵢ=59 nM) or the β^{major} promoter sequence (Kᵢ=170 nM) were too low to generate functional association. Typical data for the competitive binding experiments with 20 or 200 nM competitor (18- or 180-fold molar excess over the labeled probe, respectively) are shown in FIG. 1B. These data indicated that all of the human and mouse embryonic and fetal β-type globin gene promoters have DR elements with differential affinities for DRED in the following order: human ε distal>human ε proximal>human γ>mouse εy distal>mouse βh1>εy proximal.

The affinities of TR2 or TR4 alone and the TR2/TR4 heterodimer to the ε-globin distal DR element were compared by expressing TR2 and TR4 separately or together in the human embryonic kidney cell line 293T (in which no endogenous TR2 or TR4 is detectable by Western blots). After transfection, nuclear extracts were prepared and examined for binding to the ε distal-DR element by EMSA (FIG. 1C). Expression of TR2 alone yielded a weak signal for a DNA-protein complex that co-migrated with the authentic DRED complex isolated from MEL cells, whereas expression of TR4 alone yielded a much more stable (i.e., more robust) EMSA complex. These data suggest that expression of TR2 or TR4 alone in 293T cells generates EMSA products that result from homodimerization of the expressed receptors. When TR2 and TR4 were coexpressed to generate TR2/TR4 heterodimers (Tanabe et al., 2002), generated an even more intense signal. These data indicate that the affinities of the ε-globin distal DR element for receptor follow the order: TR2/TR4 heterodimer>TR4 homodimer>>TR2 homodimer. In other words, the affinity of the ε-globin distal DR element for the TR2 homodimer is lower than that for either the TR2/TR4 heterodimer or TR4 homodimer, whose affinities are roughly equivalent.

### Example 3

### TR2 and TR4 Forced Expression in Transgenic Mice

The present Example investigates the roles for TR2 and TR4 in β-type globin gene regulation after their transgenic forced expression.

In order to restrict expression exclusively to hematopoietic cells, the TR2 or TR4 cDNAs were placed under the control of the mouse Gata1 locus hematopoietic regulatory domain (G1-HRD), a regulatory construct that is sufficient to drive expression of the cDNAs exclusively in primitive and definitive erythroid cells (Onodera et al., 1997). The eGFP (enhanced green fluorescent protein) gene was also placed under G1-HRD control, and used to generate a fluorescent marker to reflect the specificity of transgene expression. By microinjecting the TR2 or TR4 construct, or both, together with the eGFP construct into fertilized oocytes, transgenic lines carrying the TR2 (4 lines of Tg^{TR2} mice) or TR4 (5 lines of Tg^{TR4} mice), or both (8 lines of Tg^{TR2/TR4} mice) were generated. After backcrossing the founders to wild type mice three times, each line was demonstrated to bear only a single chromosomal integration site for all of the transgenes, revealed by co-segregation after meiotic recombination.

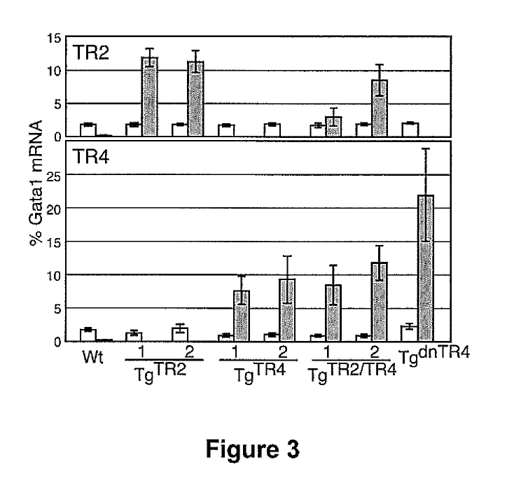

The level of transgene expression was first evaluated by eGFP fluorescence, as measured by flow cytometric analysis of adult peripheral blood. Two lines of each genotype that expressed eGFP in >80% of the erythrocytes were selected for further study. The TR2 transgene copy number varied from 1 to 7, while the TR4 transgene copy number varied between 2 and 9; as usual, transgene copy numbers did not correlate with transgene expression levels. Expression of the TgTR2 or TgTR4 transgenes in the 14.5 dpc fetal liver was determined by real time quantitative PCR; the level of Tg-derived TR2 mRNA was 1.7- to 6.8-fold higher than endogenous TR2 mRNA, while the Tg-derived TR4 mRNA levels were from 8.1 to 12.7-fold times that of endogenous TR4 mRNA (FIG. 3).

### Example 4

### The Murine Embryonic εy Gene is Uniformly Repressed, But the βH1 Gene is Differentially Regulated During Development, by TR2 and TR4

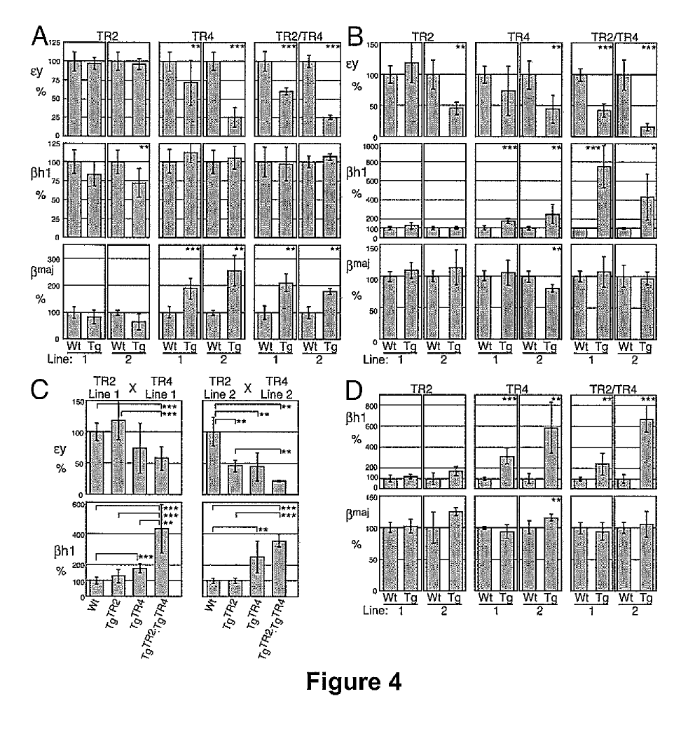

The abundance of globin mRNAs encoded by the mouse in the 9.5-dpc yolk sac, 14.5-dpc fetal liver, and adult spleen were determined by semi-quantitative RT-PCR. In the embryonic yolk sac of line 2 Tg^{TR2}, expression of the embryonic βh1 gene was inhibited to 72% of the wild type, while expression of the εy or β^{major} genes was unaffected (FIG. 4A). In yolk sac erythroid cells recovered from the two Tg^{TR4} lines, εy expression was reduced to 71% or 25% of their wild-type littermates, whereas βh1 mRNA accumulation was not affected. Unexpectedly, in both Tg^{TR4} lines, expression of the adult β^{major}-globin gene was induced by 1.8- or 2.5-fold higher than the wild type level.

In the yolk sacs of the two Tg^{TR2/TR4} lines, the εy gene was repressed to 60% or 25% of wild type levels, while expression of the adult β^{major}-globin gene was again induced to 2.1- or 1.8-fold higher than in their wild-type littermates; once again, βh1 accumulation was unaffected. The phenotypes due to concomitant transgenic expression of both TR2 plus TR4 were not discernable from the effects of TR4 alone. These data show that TR4 can repress transcription of the embryonic εy gene, and modestly activate the adult β^{major} gene in the yolk sac. In contrast, TR2 did not seem to have any regulatory effect on εy or βmajor expression, but can (acting alone) repress βh1 transcription in the embryonic yolk sac. The effects of Tg^{TR2} or Tg^{TR4} expression on β-type globin transcription in each line roughly correlated with transgene expression level (FIG. 3). Repression of the βh1 or εy genes in the Tg^{TR2} or Tg^{TR4} backgrounds at the yolk sac (primitive erythroid) stage is consistent with a role for DRED in repression of the embryonic and fetal β-type globin genes. The activation of adult β^{major} transcription upon forced TR4 transgenic expression was unpredicted, but could plausibly be a secondary consequence of promoter competition for the LCR due to coordinate εy repression.

In the fetal livers of the Tg^{TR2} lines, embryonic εy globin gene expression was repressed to 46% of wild type in one line (line 2), while βh1 and β^{major} transcription was unaffected (FIG. 4B). Tg^{TR4} forced expression resulted in εy repression to 74% or 45% of wild type. Again, to our surprise, TR4 forced expression resulted in induction of βh1 synthesis to 1.8- and 2.5-fold higher than wild type, and slightly repressed β^{major} mRNA accumulation (in one line). In the Tg^{TR2/TR4} lines, εy gene expression was repressed to 43% or 15% of their wild-type littermates, but βh1 was activated 7.6- or 4.3-fold greater than wild-type levels, but β^{major} mRNA accumulation was not affected. These data indicate that both TR2 and TR4 can repress εy transcription during the fetal liver stage. However, unexpectedly, the data also show that TR2 and TR4 can act as activators of the βh1 gene at this stage, contradicting the hypothetical role of DRED as an obligate repressor.

The Tg^{TR2/TR4} lines displayed more profound phenotypes in the fetal liver analysis than fetuses bearing only single (TR2 or TR4) transgenes, especially in activation of the βh1 gene, suggesting that the Tg^{TR2} and Tg^{TR4} may act synergistically. To test this hypothesis, Tg^{TR2} lines were crossed with Tg^{TR4} mice to produce compound transgenic fetuses in order to analyze possible genetic interactions between TR2 and TR4 (FIG. 4C). In the livers of the compound transgenic fetuses recovered from breeding Tg^{TR2} line 1 to Tg^{TR4} line 1, εy gene expression was repressed to 57% of their wild-type littermates, even though neither of the parental lines displayed any statistically significant εy repression, while βh1 transcription was activated 4.3-fold over the wild-type level, significantly higher than in either of the parental lines. Similarly, in the fetal livers of compound line 2 Tg^{TR2} and line 2 Tg^{TR4} intercrosses, εy gene expression was repressed to 22% of wild-type levels, while βh1 expression was induced to 3.5-fold times wild type. Thus, the phenotypes of the compound transgenic fetuses were again more profound than those of either parental line. These data directly demonstrate genetic synergy between the TR2 and TR4 transgenes in their effects on εy and βh1 regulation. This genetic synergy may simply reflect the higher affinity of the TR2/TR4 heterodimer for the DR element than either the TR2 or TR4 homodimer (FIG. 1). Consistent with this hypothesis, the phenotypes of the Tg^{TR4} lines were more profound than those of the Tg^{TR2} lines, in accord with the possibility that the phenotypes correlate directly with the affinities of TR2/TR4, TR4 and TR2 for the DR element. Forced transgenic expression of TR2 or TR4 also resulted in induction of βh1 transcription even in the adult spleen, but did not cause any significant change in εy- or β^{major}-globin mRNA accumulation (FIG. 4D).

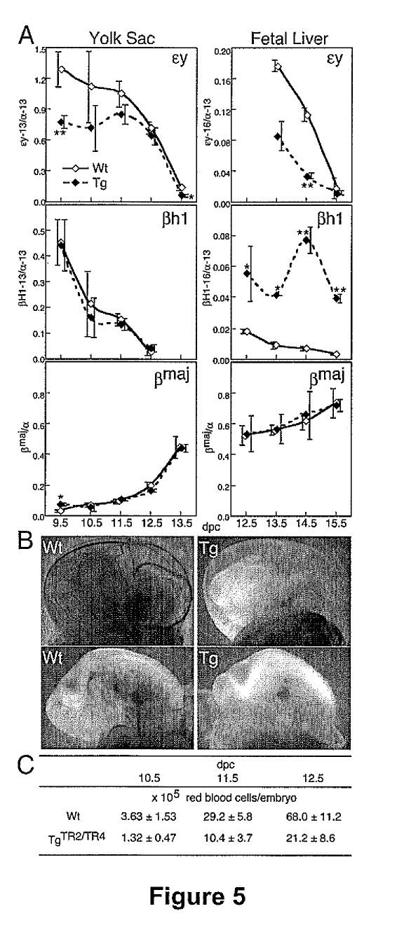

In order to understand whether the changes in β-type globin gene expression at each developmental time was the result of changes in expression level or to periodic alteration in expression during development, a complete time-course analysis of mouse β-type globin mRNA accumulation in one of the Tg^{TR2/TR4} lines (line 1) (FIG. 5A) was performed. The data show that Tg^{TR2/TR4} forced expression reduced the peak level of εy transcription in the yolk sac, and significantly accelerated the timing of εy silencing in the fetal liver. In contrast, βh1 transcription was unchanged in the yolk sac but induced in definitive fetal liver erythroid cells, causing a significant developmental delay in silencing of the βh1 gene. β^{major} transcription was unaffected throughout development, except in the 9.5 dpc yolk sac, suggesting that its transient induction was secondary to repression of the εy gene.

Tg^{TR4} embryos, whether or not they co-expressed TR2, displayed a pronounced yet transient anemia from 10.5 to 12.5 dpc in comparison to wild-type littermates (FIG. 5B). TgTR2/TR4 line 2 embryos accumulated only about one third the number of erythrocytes as their wild-type littermates during this period (FIG. 5C). The anemia was essentially fully recovered by 14.5 dpc, and all transgenic pups were born in the expected Mendelian distribution, indicating that only late primitive erythropoiesis was affected by TR2 and TR4 forced expression. This transient embryonic anemia may be due to the premature repression of εy transcription in the yolk sac, since εy is the most abundant β-type globin at this developmental stage (Fantoni et al., 1967).

Discussion:

The genetic analysis examining Tr2 or Tr4 knockout mice provides in vivo evidence for repression of the mouse βh1 gene by TR2 and TR4 in definitive erythroid cells. In contrast, erythroid-specific forced expression of wild type TR4 repressed the mouse embryonic εy-globin gene in both primitive and definitive erythroid cells, but activated the embryonic βh1 gene only in definitive erythroid cells. TR2 forced expression exerted only minor effects by comparison to TR4; TR2 repressed the εy gene only in definitive erythroid cells, and very modestly activated the βh1 gene, and only in adult spleen cells. However, co-expression of TR2 and TR4 in fetal liver erythroid cells significantly enhanced the effects of TR4 alone on both of the endogenous murine genes, indicating a strong genetic synergy between the TR2 and TR4 transgenes. Regardless of the mechanism of action, the TR2/TR4 heterodimer binds more strongly than the TR4 homodimer, and both have much greater avidity for the globin promoter DR sites than the TR2 homodimer. The unexpected activation of the βh1 gene by TR2 or TR4 forced expression seemed inconsistent with the observed modest induction of the βh1 gene in the Tr2 or Tr4 null mutant mice, but these data suggest that TR2 and TR4 can both repress and activate the βh1 gene in a context dependent manner, in keeping with the dual regulatory capacity common to most nuclear receptors (Glass and Rosenfeld, 2000).

Based on these observations, the effects of forced co-expression of TR2 and TR4 on human β-type globin gene expression by breeding to wild type human β-globin YAC transgenes was analyzed.

### Example 5

### Human ε-Globin Transcription is Repressed, and γ-Globin Transcription is Induced, in TR2 and TR4 Transgenic Mice

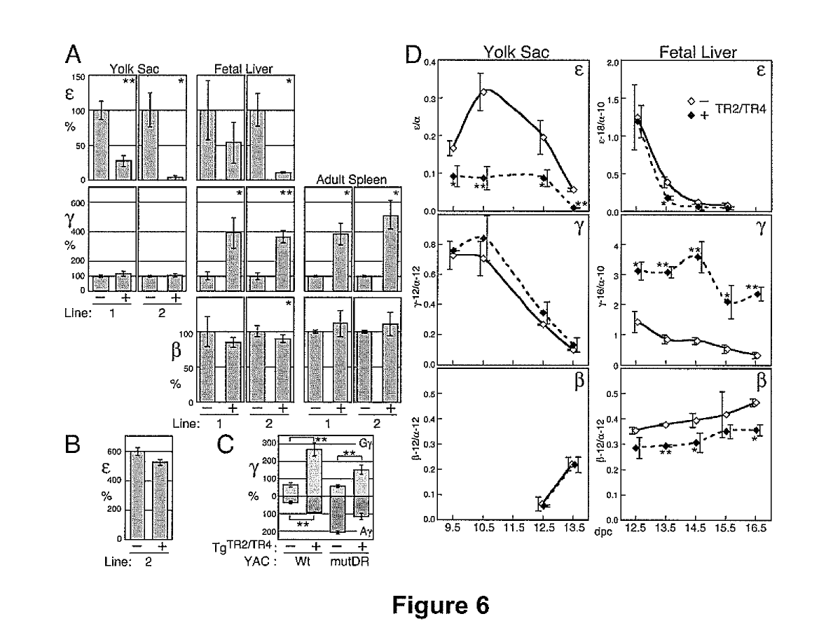

The effects of transgenic TR2/TR4 expression on human β-type globin transcription were analyzed by breeding a wild type human β-globin YAC transgene (Tg^{βYAC}) to the Tg^{TR2/TR4} lines to generate Tg^{TR2/TR4}:Tg^{βYAC} compound transgenic animals (FIG. 6A). The abundance of human β-type globin mRNAs in the 10.5 dpc yolk sac, 15.5 dpc fetal liver or adult spleen were determined by semi-quantitative RT-PCR and once again normalized to endogenous mouse α-globin mRNA abundance.

In the embryonic yolk sac, forced transgenic expression of TR2/TR4 repressed the human ε-globin gene to only 27% (line 1) or 3% (line 2) of the levels observed in their wild type (Tg^{βYAC}) littermates, but did not cause any significant change in human fetal γ- or adult β-globin transcription at the primitive stage. In the fetal liver, Tg^{TR2/TR4} expression repressed embryonic ε-globin transcription to 54% (line 2) or 10% (line 1) of wild-type, and in contrast, activated γ-globin expression to 3.9- or 3.6-fold greater than that of their Tg^{βYAC} littermates (precisely as observed at the same stage in the mouse orthologue, βh1) but also slightly repressed adult β-globin expression in line 2. In the adult spleen, γ-globin expression was also induced by 3.9- or 5.1-fold, but neither ε-globin nor β-globin accumulation was affected. These data indicate that gene-selective bi-directional regulation by TR2 and TR4 is conserved between the mouse and human β-type globin embryonic and fetal orthologues: between εy and ε, as well as between βh1 and γ.

The effects of Tg^{TR2/TR4} on the mutant human β-globin YAC transgenes that lack DR sites in either the ε- or Aγ-globin promoters (Tanimoto et al., 2000; Omori et al., 2005) were analyzed in order to determine whether or not the effects of forced TR2/TR4 expression on the embryonic and fetal β-type globin genes described above were mediated through their DR elements. One transgenic line bears a mutant human β-globin YAC that with nine nucleotide substitutions in the ε-globin gene promoter that abolishes both of its promoter DR elements (Bepsi) (Tanimoto et al., 2000). This YAC mutant (Tg^{BEPSI}) was bred to Tg^{TR2/TR4} line 2, and expression of the mutant ε-globin gene in the 10.5-dpc yolk sac was determined. These data show that repression of the ε-globin gene observed in the wild type YAC was abrogated by the DR site mutations in Tg^{BEPSI} (FIG. 6B). A second transgenic line was examined bearing a different mutant YAC transgene: mutDR with four nucleotide substitution in the Aγ-globin gene promoter that specifically abolishes its DR1 element (Omori et al., 2005). Tg^{mutDR} was bred to Tg^{TR2/TR4} line 2, and expression of the (unmodified) Gγ-globin gene (as the internal control) and the mutant Aγ-globin gene were individually quantified (Omori et al, 2005). While in the Tg^{βYAC} both the Gγ and Aγ genes were activated in the Tg^{TR2/TR4} background (FIG. 6C), only the Gγ gene (bearing an intact DR element) in the Tg^{mutDR} YAC was activated. These data demonstrate that the effects of Tg^{TR2/TR4} on the ε-globin and γ-globin genes is a direct effect elicited through TR2/TR4 binding to the DR elements in the ε- and γ-globin promoters.

Finally, a complete time-course analysis of human β-type globin transcription in the Tg^{TR2/TR4}:Tg^{βYAC} compound transgenic mice was performed. These data show that elevated TR2/TR4 expression reduced the peak level of ε-globin transcription in the yolk sac and slightly accelerated ε transcriptional silencing. In contrast, fetal γ-globin expression in the yolk sac was not significantly affected, but was induced in the fetal liver, causing a significant delay in silencing of the γ gene in definitive erythroid cells. Adult β-globin mRNA accumulation was unchanged in the yolk sac, but was repressed by 20% in the fetal liver samples. Repression of human adult β-globin synthesis in the fetal liver was unpredicted, but may be explained as a secondary consequence of activation of the γ-globin gene via promoter competition for LCR (Choi and Engel, 1988; Fraser et al., 2002).

Discussion:

The effects of TR2/TR4 forced expression were analogous to their effects on their mouse orthologues; the embryonic ε-globin gene was prematurely repressed in both primitive and definitive erythroid cells, but the fetal γ-globin gene was activated only in definitive erythroid cells. These data signify an important role for TR2 and TR4 in silencing of the ε-globin gene and hence in ε- to γ-globin gene switching.

Based on these observations, the effects of forced co-expression of TR2 and TR4 on human β-type globin gene expression by breeding to DR mutant human β-globin YAC transgenes was analyzed.

### Example 6

### The Human ε- and γ-Globin Genes are Activated by a Dominant Negative TR4 Mutant

The roles of TR2 and TR4 in human β-type globin gene regulation were further explored by employing a dominant negative TR4 (dnTR4) mutant.

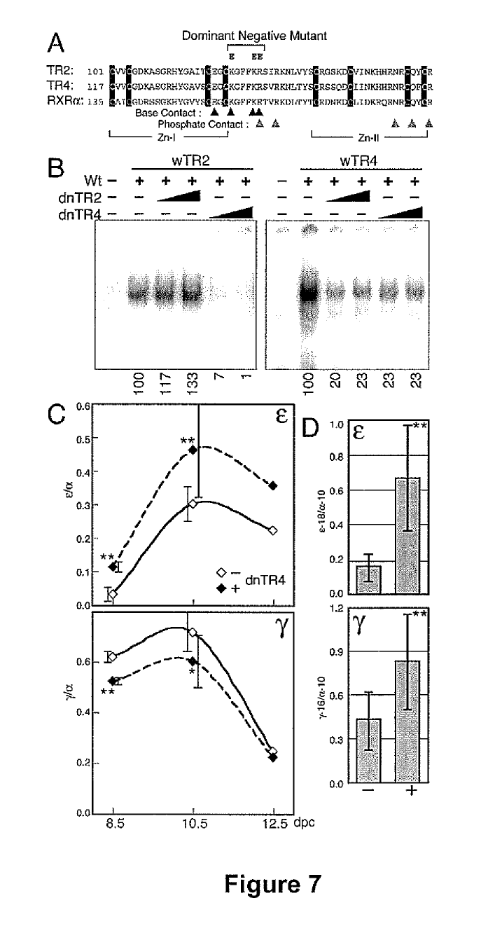

A dominant negative (dn) TR4 mutant was developed for functional analyses of TR2 and TR4 effects in regulating the β-type globin genes. The crystal structure of the DNA binding domain of RXRα, a member of the same subfamily as TR2 and TR4 in the nuclear receptor superfamily (Laudet, 1997), has been resolved, and the amino acid residues that make sequence-specific base and phosphate backbone contacts in complex with the DR1 or DR2 target elements were identified (Zhao et al., 2000) (FIG. 7A). Based on those structures, the amino acid residues in TR2 and TR4 which would make base or phosphate contacts to the DR elements were predicted. Three amino acid substitutions for those residues (Lys or Arg to Glu) were introduced to generate potential dnTR2 and dnTR4 mutants that should be defective in DNA binding activity, but should retain other activities (e.g., homo- or heterodimer formation and interaction with possible co-regulators).

The putative dn mutants were expressed in 293T cells to test their DNA binding activities (FIG. 1C). After transient transfection, nuclear extracts were prepared and examined for binding of the force-expressed putative dn receptors to the human ε distal DR element. Expression of either the TR2 or TR4 putative dn mutant yielded no DNA/protein complex, although the mutant proteins were detected in the nuclear extracts by Western blotting, indicating that the mutants are abundantly expressed in the transfected cells but are totally devoid of DNA binding activity. The TR2 or TR4 mutant proteins were next co-expressed with the wild-type proteins to confirm whether the mutants could, in fact, serve as dominant negative receptors (FIG. 7B). The DNA binding activity of TR2 was abolished by co-expression with the mutant TR4 protein, but was not affected by expression of the TR2 mutant. In contrast, the binding activity of TR4 was diminished by about 80% after co-expression with either the TR2 or TR4 mutant receptors. These data clearly demonstrate that the TR4 mutant serves as a bona fide dominant negative receptor isoform that blocks the DNA binding activity of both TR2 and TR4, while the TR2 mutant only blocks the activity of TR4.

Transgenic mice expressing the dnTR4 mutant in erythroid cells were generated, again employing the Gata-1 locus hematopoietic regulatory domain to direct tissue-restricted expression of the transgene. The mutant dnTR4 construct was co-injected with G1-HRD/eGFP into fertilized oocytes. After backcrossing the founder transgenic mice, lines expressing the dnTR4 transgene (and in which eGFP expression was documented in more than 90% of the adult blood cells) were bred to Tg^{βYAC} transgenic mice. Expression of Tg^{dnTR4} transgenes in the 14.5 dpc fetal liver was determined by real time quantitative PCR; the level of dnTR4 mRNA was 10.7- or 7.9-fold higher than endogenous TR2 and TR4 mRNA, respectively (FIG. 3).

A time-course analysis during development of human ε- and γ-globin mRNA accumulation in YAC transgenic mice also expressing the dnTR4 allele was then conducted (FIG. 7C). The data show that the peak of ε-globin mRNA accumulation in the yolk sac was induced upon concomitant expression of the dnTR4 mutant to 150% of Tg^{βYAC} (alone) littermates, and that ε-globin transcription in the fetal liver (FIG. 7D) was induced 4.2-fold over comparable littermates lacking dnTR4. In contrast, γ-globin mRNA accumulation was essentially unchanged as compared to YAC-only littermates in yolk sac erythroid cells, while γ, like ε, was clearly induced in the fetal liver. These data underscore the earlier conclusions (from the transgenic TR2 and TR4 gain of function experiments) that TR2/TR4 is an obligate repressor of the human embryonic ε-globin gene in vivo at both the primitive and definitive erythroid cell stages, and show that TR2/TR4 functions as a definitive stage-specific repressor of the fetal γ-globin genes.

Discussion:

Erythroid-specific expression of the dnTR4 mutant activated transgenic embryonic ε-globin in both primitive and definitive erythroid cells, consistent with the results of wild type TR2/TR4 forced expression, providing direct genetic evidence that TR2/TR4 is a bona fide repressor of the human embryonic ε-globin gene. In contrast, the human fetal γ-globin gene was only slightly repressed during the yolk sac stage, but induced at the fetal liver stage, indicating that TR2/TR4 differentially regulates the temporal control over γ-globin transcription during development. These data are consistent with the observed induction of the βh1 gene, the mouse orthologue of human y, in the Tr2 or Tr4 null mutant mice. The data taken together demonstrate that TR2/TR4 is a definitive-stage specific repressor of the γ-globin gene, and that TR2/TR4 plays a critical role in the sequential developmental stage-specific repression of the 8ε- and γ-globin genes that is a fundamental property inherent in 13-globin gene switching.

The repression of the embryonic mouse εy- and human ε-globin genes after forced transgenic expression of TR2/TR4, when considered along with the induction of the ε-globin gene in the compound TR2/TR4 homozygous null fetuses and after breeding to the dnTR4 mutant, provides compelling genetic evidence that TR2/TR4 is a bona fide repressor of the mouse and human embryonic globin genes. In contrast, the absence of repression of the murine embryonic βh1- and human fetal γ-globin genes in primitive erythroid cells, and the unexpected induction of those genes in definitive erythroid cells upon TR2 or TR4 forced expression, superficially contradicts the proposed repressor function of TR2 and TR4 for the βh1- and γ-globin genes deduced from the loss-of-function and dominant-negative analyses.

### LITERATURE CITED

Behringer, R. R., Ryan, T. M., Palmiter, R. D., Brinster, R. L., and Townes, T. M. (1990). Human γ- to β-globin gene switching in transgenic mice. Genes Dev 4, 380-389.
Berry, M., Grosveld, F., and Dillon, N. (1992). A single point mutation is the cause of the Greek form of hereditary persistence of fetal haemoglobin. Nature 358, 499-502.
Bunn, H. F. (2001). Human hemoglobins: sickle hemoglobin and other mutants. in The molecular basis of blood diseases, 3rd edition. Stamatoyannopoulos, G., Majerus, P. W., Perlumtter, R. M., and Varmus, H., eds. (Philadelphia, Pa.: W.B. Saunders), pp. 227-273.
Cavailles, V., Dauvois, S., L'Horset, F., Lopez, G., Hoare, S., Kushner, P. J., and Parker, M. G. (1995). Nuclear factor RIP140 modulates transcriptional activation by the estrogen receptor. Embo J 14, 3741-3751.
Chen, Y. T., Collins, L. L., Uno, H., and Chang, C. (2005). Deficits in motor coordination with aberrant cerebellar development in mice lacking testicular orphan nuclear receptor 4. Mol Cell Biol 25, 2722-2732.
Cheng, Y., and Prusoff, W. H. (1973). Relationship between the inhibition constant (Ki) and the concentration of inhibitor which causes 50 percent inhibition (150) of an enzymatic reaction. Biochem Pharmacol 22, 3099-3108.
Choi, O. R., and Engel, J. D. (1988). Developmental regulation of β-globin gene switching. Cell 55, 17-26.
Collins, L. L., Lee, Y. F., Heinlein, C. A., Liu, N. C., Chen, Y. T., Shyr, C. R., Meshul, C. K., Uno, H., Platt, K. A., and Chang, C. (2004). Growth retardation and abnormal maternal behavior in mice lacking testicular orphan nuclear receptor 4. Proc Natl Acad Sci U S A 101, 15058-15063.
Dillon, N., and Grosveld, F. (1991). Human γ-globin genes silenced independently of other genes in the β-globin locus. Nature 350, 252-254.
Enver, T., Raich, N., Ebens, A. J., Papayannopoulou, T., Costantini, F., and Stamatoyannopoulos, G. (1990). Developmental regulation of human fetal-to-adult globin gene switching in transgenic mice. Nature 344, 309-313.
Fantoni, A., Bank, A., and Marks, P. A. (1967). Globin composition and synthesis of hemoglobins in developing fetal mice erythroid cells. Science 157, 1327-1329.
Filipe, A., Li, Q., Deveaux, S., Godin, I., Romeo, P. H., Stamatoyannopoulos, G., and Mignotte, V. (1999). Regulation of embryonic/fetal globin genes by nuclear hormone receptors: a novel perspective on hemoglobin switching. Embo J 18, 687-697.
Glass, C. K., and Rosenfeld, M. G. (2000). The coregulator exchange in transcriptional functions of nuclear receptors. Genes Dev 14, 121-141.
Hanscombe, O., Whyatt, D., Fraser, P., Yannoutsos, N., Greaves, D., Dillon, N., and Grosveld, F. (1991). Importance of globin gene order for correct developmental expression. Genes Dev 5, 1387-1394.
Huisman, T. H. J., Carver, M. F. H., and Baysal, E. (1997). A syllabus of thalassemia mutations (Augusta, Ga.: The Sickle Cell Anemia Foundation).
Jiang, G., Nepomuceno, L., Yang, Q., and Sladek, F. M. (1997). Serine/threonine phosphorylation of orphan receptor hepatocyte nuclear factor 4. Arch Biochem Biophys 340, 1-9.
Laudet, V. (1997). Evolution of the nuclear receptor superfamily: early diversification from an ancestral orphan receptor. J Mol Endocrinol 19, 207-226.
Lee, C. H., Chinpaisal, C., and Wei, L. N. (1998). Cloning and characterization of mouse RIP140, a corepressor for nuclear orphan receptor TR2. Mol Cell Biol 18, 6745-6755.
Lee, Y. F., Lee, H. J., and Chang, C. (2002). Recent advances in the TR2 and TR4 orphan receptors of the nuclear receptor superfamily. J Steroid Biochem Mol Biol 81, 291-308.
Li, J., Noguchi, C. T., Miller, W., Hardison, R., and Schechter, A. N. (1998). Multiple regulatory elements in the 5′-flanking sequence of the human ε-globin gene. J Biol Chem 273, 10202-10209.
Li, Q., Blau, C. A., Clegg, C. H., Rohde, A., and Stamatoyannopoulos, G. (1998). Multiple ε-promoter elements participate in the developmental control of ε-globin genes in transgenic mice. J Biol Chem 273, 17361-17367.
Li, Q., Clegg, C., Peterson, K., Shaw, S., Raich, N., and Stamatoyannopoulos, G. (1997). Binary transgenic mouse model for studying the trans control of globin gene switching: evidence that GATA-1 is an in vivo repressor of human ε gene expression. Proc Natl Acad Sci USA 94, 2444-2448.
Li, Q., Duan, Z. J., and Stamatoyannopoulos, G. (2001). Analysis of the mechanism of action of non-deletion hereditary persistence of fetal hemoglobin mutants in transgenic mice. Embo J 20, 157-164.
Magram, J., Chada, K., and Costantini, F. (1985). Developmental regulation of a cloned adult β-globin gene in transgenic mice. Nature 315, 338-340.
Medin, J. A., Minucci, S., Driggers, P. H., Lee, I. J., and Ozato, K. (1994). Quantitative increases in DNA binding affinity and positional effects determine 9-cis retinoic acid induced activation of the retinoid X receptor β homodimer. Mol Cell Endocrinol 105, 27-35.
Mu, X., Lee, Y. F., Liu, N. C., Chen, Y. T., Kim, E., Shyr, C. R., and Chang, C. (2004). Targeted inactivation of testicular nuclear orphan receptor 4 delays and disrupts late meiotic prophase and subsequent meiotic divisions of spermatogenesis. Mol Cell Biol 24, 5887-5899.
Nagy, A., Gertsenstein, M., Vintersten, K., and Behringer, R. (2003). Manipulating the mouse embryo, 3rd edn (Cold Spring Harbor, N.Y.: Cold Spring Harbor Laboratory Press).
Omori, A., Tanabe, O., Engel, J. D., Fukamizu, A., and Tanimoto, K. (2005). Adult stage γ-globin silencing is mediated by a promoter direct repeat element. Mol Cell Biol 25, 3443-3451.
Onodera, K., Takahashi, S., Nishimura, S., Ohta, J., Motohashi, H., Yomogida, K., Hayashi, N., Engel, J. D., and Yamamoto, M. (1997). GATA-1 transcription is controlled by distinct regulatory mechanisms during primitive and definitive erythropoiesis. Proc Natl Acad Sci USA 94, 4487-4492.
Perkins, A. C., Gaensler, K. M., and Orkin, S. H. (1996). Silencing of human fetal globin expression is impaired in the absence of the adult β-globin gene activator protein EKLF. Proc Natl Acad Sci USA 93, 12267-12271.
Peters, B., Merezhinskaya, N., Diffley, J. F., and Noguchi, C. T. (1993). Protein-DNA interactions in the ε-globin gene silencer. J Biol Chem 268, 3430-3437.
Platt, O, S., Brambilla, D. J., Rosse, W. F., Milner, P. F., Castro, O., Steinberg, M. H., Klug, P. P. (1994). Mortality in sickle cell disease. Life expectancy and risk factors for early death. N Engl J Med 330, 1639-1644.
Raich, N., Clegg, C. H., Grofti, J., Romeo, P. H., and Stamatoyannopoulos, G. (1995). GATA1 and YY1 are developmental repressors of the human ε-globin gene. Embo J 14, 801-809.
Raich, N., Enver, T., Nakamoto, B., Josephson, B., Papayannopoulou, T., and Stamatoyannopoulos, G. (1990). Autonomous developmental control of human embryonic globin gene switching in transgenic mice. Science 250, 1147-1149.
Raich, N., Papayannopoulou, T., Stamatoyannopoulos, G., and Enver, T. (1992). Demonstration of a human ε-globin gene silencer with studies in transgenic mice. Blood 79, 861-864.
Ronchi, A., Berry, M., Raguz, S., Imam, A., Yannoutsos, N., Ottolenghi, S., Grosveld, F., and Dillon, N. (1996). Role of the duplicated CCAAT box region in γ-globin gene regulation and hereditary persistence of fetal haemoglobin. Embo J 15, 143-149.
Scully, K. M., Jacobson, E. M., Jepsen, K., Lunyak, V., Viadiu, H., Carriere, C., Rose, D. W., Hooshmand, F., Aggarwal, A. K., and Rosenfeld, M. G. (2000). Allosteric effects of Pit-1 DNA sites on long-term repression in cell type specification. Science 290, 1127-1131.
Shyr, C. R., Collins, L. L., Mu, X. M., Platt, K. A., and Chang, C. (2002). Spermatogenesis and testis development are normal in mice lacking testicular orphan nuclear receptor 2. Mol Cell Biol 22, 4661-4666.
Stamatoyannopoulos, G., and Grosveld, F. (2001). Hemoglobin Switching. In The molecular basis of blood diseases, 3rd edn. Stamatoyannopoulos, G., Majerus, P. W., Perlumtter, R. M., and Varmus, H., eds. (Philadelphia, Pa.: W.B. Saunders), pp. 135-182.
Steinberg, M. H., and Rodgers, G. P. (2001). Pharmacologic modulation of fetal hemoglobin. Medicine (Baltimore) 80, 328-344.
Stuart, M. J., and Nagel, R. L. (2004). Sickle-cell disease. Lancet 364, 1343-1360.
Tanabe, O., Katsuoka, F., Campbell, A. D., Song, W., Yamamoto, M., Tanimoto, K., and Engel, J. D. (2002). An embryonic/fetal β-type globin gene repressor contains a nuclear receptor TR2/TR4 heterodimer. Embo J 21, 3434-3442.
Tanimoto, K., Liu, Q., Bungert, J., and Engel, J. D. (1999). Effects of altered gene order or orientation of the locus control region on human β-globin gene expression in mice. Nature 398, 344-348.
Tanimoto, K., Liu, Q., Grosveld, F., Bungert, J., and Engel, J. D. (2000). Context-dependent EKLF responsiveness defines the developmental specificity of the human ε-globin gene in erythroid cells of YAC transgenic mice. Genes Dev 14, 2778-2794.
Townes, T. M., Lingrel, J. B., Chen, H. Y., Brinster, R. L., and Palmiter, R. D. (1985). Erythroid specific expression of human β-globin genes in transgenic mice. Embo J 4, 1715-1723.
Whitelaw, E., Tsai, S. F., Hogben, P., and Orkin, S. H. (1990). Regulated expression of globin chains and the erythroid transcription factor GATA-1 during erythropoiesis in the developing mouse. Mol Cell Biol 10, 6596-6606.
Wijgerde, M., Gribnau, J., Trimborn, T., Nuez, B., Philipsen, S., Grosveld, F., and Fraser, P. (1996). The role of EKLF in human β-globin gene competition. Genes Dev 10, 2894-2902.
Yang, Y., Wang, X., Dong, T., Kim, E., Lin, W. J., and Chang, C. (2003). Identification of a novel testicular orphan receptor-4 (TR4)-associated protein as repressor for the selective suppression of TR4-mediated transactivation. J Biol Chem 278, 7709-7717.
Zhao, Q., Chasse, S. A., Devarakonda, S., Sierk, M. L., Ahvazi, B., and Rastinejad, F. (2000). Structural basis of RXR-DNA interactions. J Mol Biol 296, 509-520.

We claim:

1. A method of identifying a compound that stimulates expression of a gene product in a definitive erythroid cell, the method comprising the step of measuring expression of a gene product in the absence and presence of a candidate substance, TR2, and TR4 with a γ-globin gene promoter sequence, wherein said gene product is encoded by a polynucleotide operatively linked to the γ-globin gene promoter sequence and the TR2 and TR4 form a heterodimer and bind the promoter sequence in the absence of the candidate substance and wherein an increase in gene product expression in the presence of the candidate substance compared to gene product expression in the absence of the candidate substance identifies the candidate substance a compound that stimulates expression of the gene product.

2. The method of claim 1 wherein the candidate substance is selected from the group consisting of a small molecule, a peptide, a polypeptide, a synthetic compound, and a naturally-occurring compound.

3. The method of claim 2, wherein the candidate substance is an antibody or an antigen binding fragment or derivative thereof.

4. The method of claim 1, which is carried out ex vivo.

5. The method of claim 1, which is carried out in vivo.

6. The method of claim 1, wherein the gene product is γ-globin.

7. The method of claim 1 wherein the gene product is a polypeptide encoded by a polynucleotide, which does not encode γ-globin, operatively-linked to the γ-globin gene promoter.

8. A compound that stimulates expression of a gene product identified by the method of claim 1.

9. A composition comprising the compound of claim 8.

10. A pharmaceutical composition comprising the substance of claim 8 and a pharmaceutically acceptable carrier, diluent, or excipient.

11. A method of identifying a compound that stimulates expression of a gene product in a definitive erythroid cell, the method comprising the step of measuring expression of a gene product in the absence and presence of a candidate substance and a TR2/TR4 heterodimer with a γ-globin gene promoter sequence, wherein said gene product is encoded by a polynucleotide operatively linked to the γ-globin gene promoter sequence and the TR2/TR4 heterodimer binds the promoter sequence in the absence of the candidate substance and wherein an increase in gene product expression in the presence of the candidate substance compared to gene product expression in the absence of the candidate substance identifies the candidate substance as a compound that stimulates expression of the gene product.

12. The method of claim 11, wherein the TR2/TR4 heterodimer is part of a direct repeat erythroid definitive (DRED) protein complex (DRED/TR2/TR4) and wherein the increase in gene product expression is associated with a decrease in DRED/TR2/TR4 binding to γ-globin gene promoter.

13. The method of claim 11, wherein the TR2/TR4 heterodimer is independent of a direct repeat erythroid definitive (DRED) protein complex and wherein the increase in gene product expression is associated with an increase in TR2/TR4 heterodimer binding to γ-globin gene promoter.

14. The method of claim 11, wherein the candidate substance is selected from the group consisting of a small molecule, a peptide, a polypeptide, a synthetic compound, and a naturally-occurring compound.

15. The method of claim 11, wherein the candidate substance is an antibody or an antigen binding fragment or derivative thereof.

16. The method of claim 11, which is carried out ex vivo.

17. The method of claim 11, which is carried out in vivo.

18. The method of claim 11, wherein the gene product is γ-globin.

19. The method of claim 11, wherein the gene product is a polypeptide encoded by a polynucleotide, which does not encode γ-globin, operatively-linked to the γ-globin gene promoter.

20. A compound that stimulates expression of a gene product identified by the method of claim 11.

21. A composition comprising the compound of claim 20.

22. A pharmaceutical composition comprising the substance of claim 20 and a pharmaceutically acceptable carrier, diluent, or excipient.

23. A method of identifying a compound that inhibits formation of a TR2/TR4 heterodimer, the methods comprising the step of measuring TR2/TR4 heterodimer formation in the absence and presence of a candidate substance, wherein a decrease in the formation of a TR2/TR4 heterodimer in the presence of the candidate substance compared to heterodimer formation in the absence of the candidate substance identifies the candidate substance as a compound that inhibits the formation of the TR2/TR4 heterodimer.

24. The method of claim 23, wherein the candidate substance is selected from the group consisting of a small molecule, a peptide, a polypeptide, a synthetic compound, and a naturally-occurring compound.

25. The method of claim 23, wherein the candidate substance is an antibody or an antigen binding fragment or derivative thereof.

26. The method of claim 23, which is carried out ex vivo.

27. The method of claim 23, which is carried out in vivo.

28. A compound that inhibits formation of a TR2/TR4 heterodimer identified by the method of claim 23.

29. A composition comprising the compound of claim 28.

30. A pharmaceutical composition comprising the substance of claim 28 and a pharmaceutically acceptable carrier, diluent, or excipient.

31. A method of treating a disorder associated with aberrant globin expression comprising the step of administering a therapeutically effective amount of the substance of any one of claims 8, 20, and 28.

32. A method of treating a disorder associated with aberrant globin expression comprising the step of contacting a TR2/TR4 heterodimer with an inhibitor that prevents binding of said TR2/TR4 heterodimer to a γ-globin gene promoter sequence.

33. A method of treating a disorder associated with aberrant globin expression comprising the step of contacting TR2 and/or TR4 with an inhibitor that prevents heterodimer formation.

34. The method of any one of claims 31-33, wherein the disorder is associated with expression of an abnormal β globin gene product.

35. The method of claim 34, wherein the disorder is sickle cell anemia.

36. The method of claim 34, wherein the disorder is β-thalessemia.

37. A method of treating sickle cell anemia comprising the step of administering to an individual in need a therapeutically effective amount of a compound that selectively stimulates expression of γ globin and reduces expression of β globin.
